(19) United States
(12) Patent Application Publication
(10) Pub. No.: US 2024/0034784 A1
(43) Pub. Date: Feb. 1, 2024

(54) ANTIBODIES TARGETING HUMAN CLAUDIN 18.2 AND USES THEREOF

(71) Applicant: Nanjing GenScript Biotech Co., Ltd., Nanjing (CN)

(72) Inventors: Liusong Yin, Nanjing (CN); Zhongdao Li, Nanjing (CN); Zhihui Zhao, Nanjing (CN)

(73) Assignee: Nanjing GenScript Biotech Co., Ltd., Nanjing, JS (CN)

(21) Appl. No.: 18/245,114

(22) PCT Filed: Sep. 29, 2021
(86) PCT No.: PCT/CN2021/121585

§ 371 Date: Mar. 13, 2023

(30) Foreign Application Priority Data
Sep. 30, 2020 (WO) PCT/CN2020/119648
Jan. 18, 2021 (WO) PCT/CN2021/072534

(51) Int. Cl. C07K 16/28; C12N 15/63; A61P 35/00

(52) U.S. Cl. CPC C07K 16/28; C12N 15/63; A61P 35/00; C07K 2317/565; C07K 2317/52; C07K 2317/569; C07K 2317/24; C07K 2317/31

(57) ABSTRACT

An isolated antibody, or an antigen-binding portion thereof, targeting Claudin 18.2. The antibody can be single domain antibody, or its monomeric variable domain can be fused with a heavy chain constant region, such as a human IgG1 Fc. Pharmaceutical compositions including the antibody, and methods of use of the pharmaceutical compositions in the treatment of diseases are also provided.

### CROSS REFERENCE TO RELATED APPLICATIONS

This is the U.S. National Stage of International Application No. PCT/CN2021/121585, filed Sep. 29, 2021, which was published in English under PCT Article 21(2), which in turn claims the benefit of International Application No. PCT/CN2020/119648, filed Sep. 30, 2020, and International Application No. PCT/CN2021/072534 filed Jan. 18, 2021. International Application No. PCT/CN2021/121585 is incorporated herein by reference in its entirety.

### INCORPORATION OF ELECTRONIC SEQUENCE LISTING

The electronic sequence listing, submitted herewith as a TXT file named Sequence Listing.txt (103,622 bytes), created on Mar. 8, 2023, is herein incorporated by reference in its entirety.

### BACKGROUND

Antibody therapies are approved in various jurisdictions to treat a wide range of cancers, and have significantly improved patient outcomes (Komeev et al., (2017) Cytokine 89: 127-135). Once bound to a cancer antigen, antibodies may induce antibody-dependent cell-mediated cytotoxicity, activate the complement system, or prevent a receptor from interacting with its ligand, all of which can lead to cancer cell deaths. U.S. FDA-approved antibody drugs include Alemtuzumab, Nivolumab, Rituximab and Durvalumab.

Claudins (CLDN) are a family of surface proteins forming a paracellular barrier, and control the flow of molecules between cells. To date, 24 claudin members have been reported in mammals. Different claudin members are expressed on different tissues, and their expression levels and functions have been linked to cancers.

Claudin 18 has two isoforms. Claudin 18.1 protein (or simply Claudin 18.1 or Claudin18.1) is selectively expressed on normal lung and stomach epithelia. Claudin 18.2 protein (or simply Claudin 18.2 or Claudin18.2) has a highly restricted expression pattern in normal tissues. High level expression of Claudin 18.2 have been observed in a significant proportion of gastric and pancreatic adenocarcinomas, making Claudin 18.2 promising targets for therapeutic strategies for the treatment of gastric and pancreatic adenocarcinoma.

Anti-Claudin 18.2 antibodies have been studied in the treatment of certain cancers. For example, Claudiximab (IMAB362), a chimeric anti-Claudin 18.2 IgG1 antibody developed by Ganymed Pharmaceuticals AG, has been investigated in clinical trials for treating advanced gastroesophageal and pancreatic cancers.

There is a need for additional anti-Claudin 18.2 antibodies with desirable therapeutic efficacies.

### SUMMARY OF THE INVENTION

In one aspect, the present disclosure provides an isolated antibody, or an antigen-binding portion thereof, which comprises a monomeric variable domain comprising a CDR1 region, a CDR2 region, and a CDR3 region comprising the amino acid sequences of:
(1) SEQ ID NOs: 52, 53, and 54, respectively;
(2) SEQ ID NOs: 55, 56, and 57, respectively;
(3) SEQ ID NOs: 58, 59, and 60, respectively;
(4) SEQ ID NOs: 61, 62, and 63, respectively;
(5) SEQ ID NOs: 64, 65, and 66, respectively;
(6) SEQ ID NOs: 76, 77, and 78, respectively;
(7) SEQ ID NOs: 82, 83, and 84, respectively;
(8) SEQ ID NOs: 85, 86, and 87, respectively;
v(9) SEQ ID NOs: 88, 89, and 90, respectively;
(10) SEQ ID NOs: 91, 92, and 93, respectively;
(11) SEQ ID NOs: 94, 95, and 96, respectively;
(12) SEQ ID NOs: 103, 104, and 105, respectively;
(13) SEQ ID NOs: 106, 107, and 108, respectively;
(14) SEQ ID NOs: 112, 113, and 114, respectively;
(15) SEQ ID NOs: 115, 116, and 117, respectively;
(16) SEQ ID NOs: 121, 122, and 123, respectively;
(17) SEQ ID NOs: 124, 125, and 126, respectively;
(18) SEQ ID NOs: 127, 128, and 129, respectively;
(19) SEQ ID NOs: 133, 134, and 135, respectively;
(20) SEQ ID NOs: 136, 137, and 138, respectively;
(21) SEQ ID NOs: 139, 140, and 141, respectively;
(22) SEQ ID NOs: 142, 143, and 144, respectively;
(23) SEQ ID NOs: 145, 146, and 147, respectively;
(24) SEQ ID NOs: 148, 149, and 150, respectively;
(25) SEQ ID NOs: 151, 152, and 153, respectively;
(26) SEQ ID NOs: 154, 155, and 156, respectively;
(27) SEQ ID NOs: 157, 158, and 159, respectively;
(28) SEQ ID NOs: 160, 161, and 162, respectively;
(29) SEQ ID NOs: 163, 164, and 165, respectively;
(30) SEQ ID NOs: 169, 170, and 171, respectively;
(31) SEQ ID NOs: 172, 173, and 174, respectively;
(32) SEQ ID NOs: 175, 176, and 177, respectively;
(33) SEQ ID NOs: 178, 179, and 180, respectively;
(34) SEQ ID NOs: 181, 182, and 183, respectively;
(35) SEQ ID NOs: 184, 185, and 186, respectively;
(36) SEQ ID NOs: 190, 191, and 192, respectively;
(37) SEQ ID NOs: 193, 194, and 195, respectively;
(38) SEQ ID NOs: 196, 197, and 198, respectively; or
(39) SEQ ID NOs: 199, 200, and 201, respectively;

or a variant of any of the above comprising up to about 3 amino acid substitutions (such as 1, 2, or 3 amino acid substitutions) in any one or more of the CDR1, CDR2, and CDR3.

In some embodiments of the antibody or antigen-binding portion thereof, the CDR1 region, the CDR2 region, and the CDR3 region comprising the amino acid sequences of:
SEQ ID NOs: 64, 65, and 66, respectively;
SEQ ID NOs: 76, 77, and 78, respectively;
SEQ ID NOs: 124, 125, and 126, respectively;
SEQ ID NOs: 136, 137, and 138, respectively;
SEQ ID NOs: 145, 146, and 147, respectively;
SEQ ID NOs: 175, 176, and 177, respectively; or
SEQ ID NOs: 199, 200, and 201, respectively;

or a variant of any of the above comprising up to about 3 amino acid substitutions (such as 1, 2, or 3 amino acid substitutions) in any one or more of the CDR1, CDR2, and CDR3.

In some embodiments, the CDR1 region, the CDR2 region, and the CDR3 region comprising the amino acid sequences of one of the following: SEQ ID NOs: 64, 65, and 66, respectively; SEQ ID NOs: 76, 77, and 78, respectively; SEQ ID NOs: 124, 125, and 126, respectively; SEQ ID NOs: 136, 137, and 138, respectively; SEQ ID NOs: 145, 146, and 147, respectively; SEQ ID NOs: 175, 176, and 177, respectively; or SEQ ID NOs: 199, 200, and 201, respectively.

In some embodiments, the antibody or an antigen-binding portion thereof comprises the amino acid sequence of a CDR1, a CDR2, and a CDR3 within a monomeric variable domain having the amino acid sequence set forth in any of SEQ ID NOs: 1, 2, 3, 4, 5, 9, 11, 12, 13, 14, 18, 19, 21, 22, 24, 25, 26, 28, 29, 30, 31, 32, 33, 34, 35, 36, 37, 38, 40, 41, 42, 43, 44, 45, 47, 48, 49, and 50.

In some embodiments, the antibody or an antigen-binding portion thereof specifically binds to Claudin 18.2, preferably human Claudin 18.2.

In some embodiments, the antibody or antigen-binding portion thereof is a single domain antibody (sdAb) or a V_{H}H domain.

In some embodiments, the antibody or antigen-binding portion thereof is camelid, chimeric, human or humanized.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213.

In some embodiments, the monomeric variable domain comprises an amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs:5, 9, 25, 29, 32, 42, and 202-213.

In some embodiments, the monomeric variable domain comprises an amino acid sequence selected from the group consisting of SEQ ID NOs: 5, 9, 25, 29, 32, 42, 50 and 202-213.

In another aspect, the present disclosure provides an antibody or antigen-binding portion thereof comprising a monomeric variable domain that comprises an amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213. In some embodiments, the amino acid sequence is selected from the group consisting of SEQ ID NOs: 5, 9, 25, 29, 32, 42, 50 and 202-213.

In a further aspect, the present disclosure provides a bispecific molecule, an immunoconjugate, or a chimeric antigen receptor, comprising the antibody or antigen-binding portion thereof of any of the antibody or antigen-binding portion thereof described herein.

In yet a further aspect, the present disclosure provides a nucleic acid molecule encoding the antibody or antigen-binding portion thereof of any of the antibody (or antigen-binding portion thereof) or the bispecific molecule, an immunoconjugate, or a chimeric antigen receptor of described herein. An expression vector containing the nucleic acid molecule, and a host cell containing the expression vector, are also provided.

In yet a further aspect, the present disclosure provides a pharmaceutical composition comprising (1) the antibody or antigen-binding portion thereof described herein, or the bispecific molecule, an immunoconjugate, or a chimeric antigen receptor described herein, and (2) a pharmaceutically acceptable carrier. The pharmaceutical composition can further comprise a cytotoxic agent.

In yet a further aspect, the present disclosure provides a method for treating a cancer disease in a subject, comprising administering to the subject a therapeutically effective amount of the pharmaceutical composition(s) described herein. The cancer disease can be selected from the group consisting of gastric cancer, pancreatic cancer, colon cancer, esophageal cancer, hepatic cancer, ovarian cancer, lung cancer and bladder cancer.

### BRIEF DESCRIPTION OF THE DRAWINGS

FIGS. 1A-1F (show FACS binding curves of certain embodiments of antibodies the present disclosure with human Claudin18.2 or Claudin18.1-overexpressing HEK293 cells, as compared to control antibody and negative control. FIGS. 1A-1D show results from example antibodies of the present disclosure. FIGS. 1E and 1F: IMAB362 antibody is used as positive control and PBS is used as negative control.

FIGS. 2A-2E show dose-response curves for FACS-binding assay of certain embodiments of antibodies of the present disclosure against human Claudin 18.1-overexpressing HEK293 cells.

FIGS. 3A-3F show dose-response curves for FACS-binding assay data of certain embodiments of antibodies of the present disclosure against human Claudin 18.2-overexpressing HEK293 cells.

FIGS. 4A-4E show dose-response curves for FACS-binding assay data of certain embodiments of antibodies of the present disclosure against mouse Claudin 18.2-overexpressing HEK293 cells.

FIGS. 5A-5H show CDC assay results of certain embodiments of antibodies of the present disclosure.

FIGS. 6A-6G show ADCC assay results of certain embodiments of antibodies of the present disclosure.

FIGS. 7A-7B show dose-response curves for FACS-binding assay data of certain embodiments of antibodies of the present disclosure against human Claudin 18.2-overexpressing HEK293 cells.

FIGS. 8A-8B show dose-response curves for FACS-binding assay of certain embodiments of antibodies of the present disclosure against human Claudin 18.1-overexpressing HEK293 cells.

FIGS. 9A-9C shows ADCC assay results of certain embodiments of antibodies of the present disclosure.

FIG. 10 shows CDC assay results of certain embodiments of antibodies of the present disclosure.

FIGS. 11A-11B show ADCC assay results of certain embodiments of antibodies of the present disclosure.

FIGS. 12A-12B show dose-response curves for FACS-binding assay data of certain embodiments of antibodies of the present disclosure against human Claudin 18.2-overexpressing CHO-K1 cells

### DETAILED DESCRIPTION OF EMBODIMENTS

The term “antigen-binding portion” of an antibody as used herein refers to one or more fragments of an antibody that retain the ability to bind to an antigen (e.g., a Claudin 18.2 protein). This can include a heavy chain variable region containing a single variable domain, as well as a bigger molecule that includes such a domain and another polypeptide segment chemically linked to it. Such single chain antibodies Marc also intended to be encompassed within the term “antigen-binding portion”.

An “isolated antibody” as used herein refers to an antibody that is substantially free of other antibodies having different antigenic specificities. An. isolated. antibody that specifically binds a Claudin 18.2 protein is substantially free of antibodies that do not bind to a Claudin 18.2 protein. An isolated antibody that specifically binds a human Claudin 18.2 may also specifically hind other antigens, such as human Claudin 18.1 protein or Claudin 18.2 proteins from other species. An isolated antibody can also be substantially free of other cellular material and/or chemicals.

The terms “monoclonal antibody” as used herein refer to a preparation of antibody molecules of single molecular composition.

The term “single domain antibody” or “sdAb” refers to a single antigen-binding polypeptide comprising a single monomeric variable antibody domain having three complementary determining regions (CDRs), which is capable of binding to an antigen without pairing with a corresponding CDR-containing polypeptide. In some cases, the single domain antibody is engineered from a camelid heavy chain antibodies (HCAb), and is also called the V_{H}H domain or fragment of the HCAb. The single domain antibody is a kind of antigen-binding portion of a heavy chain only antibody. The V_{H}Hs may also be known as Nanobodies. Camelid sdAb is one of the smallest known antigen binding antibody fragments (see. e.g., Hamers-Casterman et al., Nature 363:446-8 (1993); Greenberg et al., Nature 374:168-73 (1995); Hassanzadeh-Ghassabeh et al., Nanomedicine (Lond), 8:101.3-26 (2013)). As examples, particular single domain antibodies in the Examples of the present disclosure having the amino acid sequence of SEQ ID NOs: 1-50 are referred to sdAb-1 (or sdab-1), sdAb-2 (or sdab-2), sdAb-3 (or scab-3), . . . , sdAb-50 (or sdab-50).

As used herein, an antibody or molecule that “specifically binds to human Claudin 18.2” refers to an antibody or polypeptide molecule that binds to human Claudin 18.2 protein but does not substantially bind to non-Claudin 18.2 proteins.

As used herein, the term “fused protein”, “fusion protein”, “fusion antibody”, or “fused antibody” refers to an antibody molecule that include both a single domain antibody (e.g., V_{H}H) part and a IgG constant region Fe (such as IgG1 Fc region), where the two parts are chemically linked, e.g., by recombinant methods. For example, sdAbs can be directly fused to the IgG Fc region with the native hinge region of the IgG. The sdAb is located upstream, i.e., closer to the N-terminus, of the IgG Fc in the fused antibody, where the sdAb and the IgG Fc region are intervened by the hinge region. A fused antibody protein can be referenced herein as its constituent parts, e.g., sdab-1-hIgG1Fc refers to a fused protein or antibody made up by sdab-2 fused with the human IgG1 Fc region (having the amino acid sequence of SEQ ID NO:51), sdab-2-hIgG1Fc refers to a fused protein or antibody made up by sdab-2 fused with the human IgG1 Fc region, etc.

The term “camelid antibody”, as used herein, is intended to include antibodies having variable regions (or more specifically the single domain antibodies or V_{H}H fragments) in which both the framework and CDR regions are derived from camelid germline heavy chain only antibody sequences. Furthermore, if the antibody contains a constant region, the constant region can also be derived from camelid germline antibody sequences. The camelid antibodies of the invention can include amino acid residues not encoded by camelid germline antibody sequences (e.g., mutations introduced by random or site-specific mutagenesis in vitro or by somatic mutation in vivo). However, the term “camelid antibody”, as used herein, is not intended to include antibodies in which CDR sequences derived from the germline of another mammalian species have been grafted onto camelid framework sequences.

The term “chimeric antibody” refers to an antibody made by combining genetic material from a nonhuman (e.g., camelid) source with genetic material from a human being. Or more generally, a chimeric antibody is an antibody having different parts produced from genetic material of different species.

The term “humanized antibody”, as used herein, refers to an antibody from non-human (e.g., camelid) species whose protein sequences have been modified to increase similarity to antibody variants produced naturally in humans.

The terms percent “identity” as used herein in the context of two or more nucleic acids or proteins/peptides, refer to two or more sequences or subsequences that have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof.

As used herein, the term “subject” includes any human or nonhuman animal. The term “nonhuman animal” includes all vertebrates, e.g., mammals and non-mammals. In certain embodiments, the subject is a human.

The CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Another Exemplary system is Kabat.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

### Antibody Targeting Human Claudin 18.2 (or Anti-Human Claudin 18.2 Antibody)

In one aspect, the present disclosure provides an isolated antibody, or an antigen-binding portion thereof, comprising a monomeric variable domain comprising a CDR1 region, a CDR2 region, and a CDR3 region comprising the amino acid sequences of:
(1) SEQ ID NOs: 52, 53, and 54, respectively;
(2) SEQ ID NOs: 55, 56, and 57, respectively;
(3) SEQ ID NOs: 58, 59, and 60, respectively;
(4) SEQ ID NOs: 61, 62, and 63, respectively;
(5) SEQ ID NOs: 64, 65, and 66, respectively;
(6) SEQ ID NOs: 76, 77, and 78, respectively;
(7) SEQ ID NOs: 82, 83, and 84, respectively;
(8) SEQ ID NOs: 85, 86, and 87, respectively;
(9) SEQ ID NOs: 88, 89, and 90, respectively;
(10) SEQ ID NOs: 91, 92, and 93, respectively;
(11) SEQ ID NOs: 94, 95, and 96, respectively;
(12) SEQ ID NOs: 103, 104, and 105, respectively;
(13) SEQ ID NOs: 106, 107, and 108, respectively;
(14) SEQ ID NOs: 112, 113, and 114, respectively;
(15) SEQ ID NOs: 115, 116, and 117, respectively;
(16) SEQ ID NOs: 121, 122, and 123, respectively;
(17) SEQ ID NOs: 124, 125, and 126, respectively;
(18) SEQ ID NOs: 127, 128, and 129, respectively;
(19) SEQ ID NOs: 133, 134, and 135, respectively;
(20) SEQ ID NOs: 136, 137, and 138, respectively;
(21) SEQ ID NOs: 139, 140, and 141, respectively;
(22) SEQ ID NOs: 142, 143, and 144, respectively;
(23) SEQ ID NOs: 145, 146, and 147, respectively;
(24) SEQ ID NOs: 148, 149, and 150, respectively;
(25) SEQ ID NOs: 151, 152, and 153, respectively;
(26) SEQ ID NOs: 154, 155, and 156, respectively;
(27) SEQ ID NOs: 157, 158, and 159, respectively;
(28) SEQ ID NOs: 160, 161, and 162, respectively;
(29) SEQ ID NOs: 163, 164, and 165, respectively;
(30) SEQ ID NOs: 169, 170, and 171, respectively;
(31) SEQ ID NOs: 172, 173, and 174, respectively;
(32) SEQ ID NOs: 175, 176, and 177, respectively;
(33) SEQ ID NOs: 178, 179, and 180, respectively;
(34) SEQ ID NOs: 181, 182, and 183, respectively;
(35) SEQ ID NOs: 184, 185, and 186, respectively;
(36) SEQ ID NOs: 190, 191, and 192, respectively;
(37) SEQ ID NOs: 193, 194, and 195, respectively;
(38) SEQ ID NOs: 196, 197, and 198, respectively; or
(39) SEQ ID NOs: 199, 200, and 201, respectively;

or a variant of any of the above comprising up to about 3 amino acid substitutions (such as 1, 2, or 3 amino acid substitutions) in any one or more of the CDR1, CDR2, and CDR3.

In some embodiments, the isolated antibody, or an antigen-binding portion thereof, comprising a monomeric variable domain comprising a CDR1 region, a CDR2 region, and a CDR3 region comprising the amino acid sequences of:
(1) SEQ ID NOs: 52, 53, and 54, respectively;
(2) SEQ ID NOs: 55, 56, and 57, respectively;
(3) SEQ ID NOs: 58, 59, and 60, respectively;
(4) SEQ ID NOs: 61, 62, and 63, respectively;
(5) SEQ ID NOs: 64, 65, and 66, respectively;
(6) SEQ ID NOs: 76, 77, and 78, respectively;
(7) SEQ ID NOs: 82, 83, and 84, respectively;
(8) SEQ ID NOs: 85, 86, and 87, respectively;
(9) SEQ ID NOs: 88, 89, and 90, respectively;
(10) SEQ ID NOs: 91, 92, and 93, respectively;
(11) SEQ ID NOs: 94, 95, and 96, respectively;
(12) SEQ ID NOs: 103, 104, and 105, respectively;
(13) SEQ ID NOs: 106, 107, and 108, respectively;
(14) SEQ ID NOs: 112, 113, and 114, respectively;
(15) SEQ ID NOs: 115, 116, and 117, respectively;
(16) SEQ ID NOs: 121, 122, and 123, respectively;
(17) SEQ ID NOs: 124, 125, and 126, respectively;
(18) SEQ ID NOs: 127, 128, and 129, respectively;
(19) SEQ ID NOs: 133, 134, and 135, respectively;
(20) SEQ ID NOs: 136, 137, and 138, respectively;
(21) SEQ ID NOs: 139, 140, and 141, respectively;
(22) SEQ ID NOs: 142, 143, and 144, respectively;
(23) SEQ ID NOs: 145, 146, and 147, respectively;
(24) SEQ ID NOs: 148, 149, and 150, respectively;
(25) SEQ ID NOs: 151, 152, and 153, respectively;
(26) SEQ ID NOs: 154, 155, and 156, respectively;
(27) SEQ ID NOs: 157, 158, and 159, respectively;
(28) SEQ ID NOs: 160, 161, and 162, respectively;
(29) SEQ ID NOs: 163, 164, and 165, respectively;
(30) SEQ ID NOs: 169, 170, and 171, respectively;
(31) SEQ ID NOs: 172, 173, and 174, respectively;
(32) SEQ ID NOs: 175, 176, and 177, respectively;
(33) SEQ ID NOs: 178, 179, and 180, respectively;
(34) SEQ ID NOs: 181, 182, and 183, respectively;
(35) SEQ ID NOs: 184, 185, and 186, respectively;
(36) SEQ ID NOs: 190, 191, and 192, respectively;
(37) SEQ ID NOs: 193, 194, and 195, respectively;
(38) SEQ ID NOs: 196, 197, and 198, respectively; or
(39) SEQ ID NOs: 199, 200, and 201, respectively.

In some embodiments of the antibody or antigen-binding portion thereof, the CDR1 region, the CDR2 region, and the CDR3 region comprising the amino acid sequences of:
SEQ ID NOs: 64, 65, and 66, respectively;
SEQ ID NOs: 76, 77, and 78, respectively;
SEQ ID NOs: 124, 125, and 126, respectively;
SEQ ID NOs: 136, 137, and 138, respectively;
SEQ ID NOs: 145, 146, and 147, respectively;
SEQ ID NOs: 175, 176, and 177, respectively; or
SEQ ID NOs: 199, 200, and 201, respectively;

or a variant of any of the above comprising up to about 3 amino acid substitutions (such as 1, 2, or 3 amino acid substitutions) in any one or more of the CDR1, CDR2, and CDR3.

In some embodiments of the antibody or antigen-binding portion thereof, the CDR1 region, the CDR2 region, and the CDR3 region comprising the amino acid sequences of:
SEQ ID NOs: 64, 65, and 66, respectively;
SEQ ID NOs: 76, 77, and 78, respectively;
SEQ ID NOs: 124, 125, and 126, respectively;
SEQ ID NOs: 136, 137, and 138, respectively;
SEQ ID NOs: 145, 146, and 147, respectively;
SEQ ID NOs: 175, 176, and 177, respectively; or
SEQ ID NOs: 199, 200, and 201, respectively.

In some embodiments, the CDR1 region, the CDR2 region, and the CDR3 region comprising the amino acid sequences of one of the following: SEQ ID NOs: 64, 65, and 66, respectively; SEQ ID NOs: 76, 77, and 78, respectively; SEQ ID NOs: 124, 125, and 126, respectively; SEQ ID NOs: 136, 137, and 138, respectively; SEQ ID NOs: 145, 146, and 147, respectively; SEQ ID NOs: 175, 176, and 177, respectively; or SEQ ID NOs: 199, 200, and 201, respectively.

In some embodiments, the antibody or an antigen-binding portion thereof comprises the amino acid sequence of a CDR1, a CDR2, and a CDR3 within a monomeric variable domain having the amino acid sequence set forth in any of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, and 50.

In some embodiments, the antibody or an antigen-binding portion thereof specifically binds to Claudin 18.2, preferably human Claudin 18.2.

In some embodiments, the antibody or antigen-binding portion thereof is a single domain antibody (sdAb) or a V_{H}H domain, i.e., without a conventional heavy chain constant region. In some embodiments, the antibody or antigen-binding portion thereof is a single domain antibody.

In some embodiments, the antibody or antigen-binding portion thereof is camelid, chimeric, human or humanized.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1-50 and 202-213.

In some embodiments, the monomeric variable domain comprises an amino acid sequence selected from the group consisting of SEQ ID NOs: 1-50 and 202-213.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213.

In some embodiments, the monomeric variable domain comprises an amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213.

In some embodiments, the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs:5, 9, 25, 29, 32, 42, 50 and 202-213.

In some embodiments, the monomeric variable domain comprises an amino acid sequence selected from the group consisting of SEQ ID NOs: 5, 9, 25, 29, 32, 42, 50 and 202-213.

In another aspect, the present disclosure provides an antibody or antigen-binding portion thereof comprising a monomeric variable domain comprising an amino acid sequence selected from the group consisting of SEQ ID NOs: 1, 2, 3, 4, 5, 9, 11, 12, 13, 14, 15, 18, 19, 21, 22, 24, 26, 28, 29, 30, 31, 32, 33, 34, 35, 36, 37, 38, 40, 41, 42, 43, 44, 45, 47, 48, 49, 50 and 202-213. In some embodiments, the amino acid sequence is selected from the group consisting of SEQ ID NOs: 5, 9, 25, 29, 32, 42, 50 and 202-213.

In another aspect, the present disclosure provides an antibody or antigen-binding portion thereof comprising a monomeric variable domain that comprises an amino acid sequence having a at least 80%, 85%, 90%, 92%, 94%, 95%, 96%, 97%, 98%, or 99% identity to the amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213. In some embodiments, the antibody or antigen-binding portion thereof comprises a monomeric variable domain that comprises an amino acid sequence having a at least 80%, 85%, 90%, 92%, 94%, 95%, 96%, 97%, 98%, or 99% identity to the amino acid sequence selected from the group consisting of SEQ ID NOs: 5, 9, 25, 29, 32, 42, 50 and 202-213.

Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production. In some embodiments, the antibody or antigen-binding portion thereof described herein comprises a Fc region. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. In some embodiments, the constant region modifications increase the serum half-life of the antibody. In some embodiments, the constant region modifications increase or enhance ADCC and/or complement dependent cytotoxicity (CDC) of the antibody.

The antibody or antigen-binding portion thereof described herein can further comprise an IgG Fc region fused with the monomeric variable domain. For example, the IgG Fc region can be a human IgG1 Fc region comprising the amino acid sequence of SEQ ID NO: 51. In some embodiments, the antibody or antigen-binding portion thereof (e.g. sdAb) fused to the N-terminus of human IgG1 Fc region.

In a further aspect, the present disclosure provides a bispecific molecule, an immunoconjugate (or antibody drug conjugate), or a chimeric antigen receptor, comprising the antibody or antigen-binding portion thereof as described herein.

The term “bispecific molecule” refers to an antibody of the present disclosure linked to at least one other functional molecule, e.g., another peptide or protein (e.g., another antibody or ligand for a receptor). A bispecific molecule can have at least two different binding sites or target molecules, and can includes molecules that have three or more specificities.

The term “immunoconjugate” refers to an antibody of the present disclosure conjugated to a therapeutic agent, such as cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, etc. In the ADC, the antibody and therapeutic agent can be are conjugated via a cleavable linker such as a peptidyl, disulfide,

The term “chimeric antigen receptor” or “CAR” refers to an engineered receptor that grafts a defined specificity onto an immune effector cell, typically a T cell, and augments T-cell function. The new generation CAR comprises an extracellular binding domain comprising a single domain antibody, a hinge region, a transmembrane domain, and an intracellular signaling domain (mainly CD3-zeta's cytoplasmic domain, which is the primary transmitter of T cell activation signals, plus one or more co-stimulatory domains). The CARs may further have factors that enhance T cell expansion, persistence, and anti-tumor activity, such as cytokines and co-stimulatory ligands.

In yet a further aspect, the present disclosure provides a nucleic acid molecule encoding the antibody or antigen-binding portion thereof of any of the antibody (or antigen-binding portion thereof), or the bispecific molecule, an immunoconjugate, or a chimeric antigen receptor of described herein. A host cell (e.g., a CHO cell, or a lymphocytic cell, or microorganisms, such as E. coil, and fungi, such as yeast) containing an expression vector containing the nucleic acid molecule, can be used to produce antibodies of the present disclosure, preferably monoclonal antibodies.

In one embodiment, DNA encoding partial or full-length antibody of the present disclosure can be obtained by standard molecular biology techniques is inserted into one or more expression vectors such that the genes are operatively linked to transcriptional and translational regulatory sequences. The term “operatively linked” is intended to mean that an antibody gene is ligated into a vector such that transcriptional and translational control sequences within the vector serve their intended function of regulating the transcription and translation of the antibody gene. The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody encoding DNA can be inserted into the expression vector. The recombinant expression vector can encode a signal peptide that facilitates secretion of the antibody chain from a host cell. The antibody encoding DNA can be cloned into the vector such that the signal peptide is linked in-frame to the amino terminus of the antibody encoding DNA. The signal peptide can be an immunoglobulin signal peptide or a heterologous signal peptide (i.e., a signal peptide from a non-immunoglobulin protein).

For expression of sdAbs (or the sdAb-IgGFc), the expression vector(s) encoding the antibody chain is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Mammalian host cells for expressing the antibodies of the present disclosure include HEK293 cells, Chinese Hamster Ovary (CHO cells), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

The term “EC₅₀”, also known as half maximal effective concentration, refers to the concentration of an antibody or an antigen-binding portion thereof such as an anti-Claudin18.2 sdAb-Fc fusion protein or an anti-Claudin18.2 sdAb that gives half-maximal response after a specified exposure time.

In some embodiments, the antibody or the antigen-binding portion thereof (e.g., an sdAb or an sdAb-Fc fusion protein) binds Claudin18.2 (e.g., human Claudin18.2) with a half maximal effective concentration (EC50) of about 1 μM or less, about 100 nM or less, about 40 nM or less, about 20 nM or less, about 10 nM or less, about 1 nM or less, or about 0.1 nM or less. In some embodiments, the antibody or the antigen-binding portion thereof binds to human Claudin18.2 with an EC50 of about 1 μM or less, about 100 nM or less, about 40 nM or less, about 20 nM or less, about 10 nM or less, about 1 nM or less, or about 0.1 nM or less. In some embodiments, the antibody or the antigen-binding portion thereof binds human Claudin18.2 with an EC50 of about 40 nM or less. In some embodiments, the antibody or the antigen-binding portion thereof binds human Claudin18.2 with an EC50 of about 20 nM or less. In some embodiments, the antibody or the antigen-binding portion thereof binds human Claudin18.2 with an EC50 of about 10 nM or less. In some embodiments, the antibody or the antigen-binding portion thereof binds human Claudin18.2 with an EC50 of about 1 nM or less. In some embodiments, the antibody or the antigen-binding portion thereof binds human Claudin18.2 with an EC50 of about 0.1 nM or less.

### Pharmaceutical Compositions and Methods of Treatment

In another aspect, the present disclosure provides a pharmaceutical composition comprising one or more antibodies of the present invention, or the bispecific molecule, an immunoconjugate, or a chimeric antigen receptor described herein, together with a pharmaceutically acceptable carrier in accordance with conventional techniques.

The composition may comprise one or more additional pharmaceutically active ingredients, such as another antibody, a drug, e.g., a cytotoxic or anti-tumor agent. The pharmaceutical compositions of the invention also can be administered in a combination therapy with, for example, another anti-cancer agent, another anti-inflammatory agent, etc. As used herein, “pharmaceutically acceptable carrier” includes pharmaceutically acceptable carriers, excipients or stabilizers. These include but are not limited solvents, dispersion media, coatings, antibacterial and antifungal agents, isotonic and absorption delaying agents, surface active agents, thickening or emulsifying agents, solid binders, dispersion or suspension aids, solubilizers, colorants, flavoring agents, coatings, disintegrating agents, lubricants, sweeteners, preservatives, isotonic agents, and the like that are physiologically compatible. The selection of suitable carrier is within the knowledge of an artisan skilled in the art.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, e.g., intranasally, vaginally, rectally, sublingually or topically.

In another aspect, the present disclosure provides a method for treating a disease in a subject, e.g., a human or non-human mammal, comprising administering to the subject a therapeutically effective amount of the pharmaceutical composition described herein. The disease can be cancer selected from the group consisting of gastric cancer, pancreatic cancer, colon cancer, esophageal cancer, hepatic cancer, ovarian cancer, lung cancer, and bladder cancer. In certain embodiments, the cancer is gastric cancer. In certain embodiments, the cancer is pancreatic cancer. In certain embodiments, the subject is human.

In the administration of the composition to the subject, dosage regimens can be adjusted to provide the optimum desired response (e.g., a therapeutic response). Single bolus or divided doses can be administered based on the subject, the disease to be treated, etc. Dosage unit form as used herein refers to physically discrete units suited as unitary dosages for the subjects to be treated. Each unit contains a predetermined quantity of active ingredient calculated to produce the desired therapeutic effect in association with the required pharmaceutical carrier. Sustained release formulation can be used in which case less frequent administration is required.

For administration of an antibody of the present disclosure, the dosage may range from about 0.0001 to 100 mg/kg, and more usually 0.01 to 5 mg/kg, of the body weight of the subject. For example dosages can be 0.3 mg/kg body weight, 1 mg/kg body weight, 3 mg/kg body weight, 5 mg/kg body weight or 10 mg/kg body weight or within the range of 1-10 mg/kg. A suitable treatment regime can be once per week, once every two weeks, once every three weeks, once every four weeks, once a month, etc. Example dosage regimens for an anti-Claudin 18.2 antibody of the invention can include 1 mg/kg body weight or 3 mg/kg body weight via intravenous administration.

A “therapeutically effective amount” or “therapeutically effective amount” of an antibody targeting Claudin 18.2 of the invention preferably results in a decrease in severity of disease symptoms, an increase in frequency and/or duration of disease symptom-free periods, prevention or reduction of likelihood of impairment or disability due to the disease affliction, or inhibition or delaying of the progression of disease. For example, for the treatment of tumor-bearing subjects, a “therapeutically effective amount” of an antibody composition may inhibits tumor growth by at least about 20%, more preferably by at least about 40%, even more preferably by at least about 60%, and still more preferably by at least about 80% relative to untreated subjects.

### EXAMPLES

The examples below are intended to be purely exemplary of the invention and should therefore not be considered to limit the invention in any way.

### Example 1

### Generation of Anti-Claudin 18.2 sdAbs

### Animal Immunization

Llamas were immunized with recombinant human Claudin18.2-His proteins (GenScript) under current animal welfare regulations. For immunization, the antigen was administrated in PBS solution or formulated as an emulsion with CFA (Complete Freund's adjuvant; primary immunization) or IFA (incomplete Freund's adjuvant; boost immunizations). The antigen was administered subcutaneously at the neck. Each animal received 6 injections of the emulsion, including the primary immunization with 200 μg of antigen in CFA emulsion, the subsequent 3 boosts with 100 μg of antigen in IFA emulsion at one-week interval and the following 2 injections with 50 μg of antigen in IFA emulsion at one-week interval. After multiple rounds of immunization, a blood sample of 200 ml was collected. Peripheral blood lymphocytes (PBLs), as the genetic source of the llama heavy chain antibodies (HCAbs) were isolated from the blood sample by gradient centrifugation.

### Phage Display Library Construction

Total RNA was extracted from the isolated lymphocytes as described above using TRIZOL reagent (Thermo Fisher Scientific, cat #15596026) according to the manufacturer's protocol, which was used as starting material for RT-PCR to amplify sdAb encoding gene fragments. For gene amplification, the isolated RNA was reverse transcribed into cDNA with an oligo (dT)₂₀ primer using PRIMESCRIPT 1^{st} strand cDNA synthesis kit (Takara, cat #6110A) according to the manufacturer's protocol. Six forward and two reverse specific degenerate primers were designed to amplify the V_{H}H fragments.

The variable regions of the V_{H}H fragments were amplified by two-step PCR methods. The DNA products of the first PCR were used as templates in the second PCR. The amplified second PCR products containing V_{H}H PCR fragments were gel purified and enzyme digested followed by insertion into phagemid plasmids. The recombinant plasmids were transferred into E. coli cells by electroporation, which generated the phage display library with size more than 1×10⁹. The library phage was prepared according to a standard protocol and stored after filter sterilization at −80° C. as stock.

### Phage Display Panning

The constructed phage library was first counter-screened with CHO-K1 cells overexpressing human Claudin18.1 followed by panning with CHO-K1 cells overexpressing human Claudin18.2 using a standard procedure developed by GenScript. Two rounds of selection was performed and each selection output was analyzed for enrichment factor (number of phage present in elution relative to control), diversity and percentage of Claudin18.2 positive clones (FACS). Based on these parameters the best selections were chosen for further screening, which was subcloned as a pool into a soluble expression vector for high-throughput screening. In frame with the sdAb coding sequence, the vector coded for a C-terminal His-Tag. Colonies were picked and grown in 96 deep well plates (1 ml volume) and induced by adding IPTG and 0.1% Triton for sdAb expression in the supernatant.

Anti-Claudin18.2 Specific sdAb Leads Identification by FACS

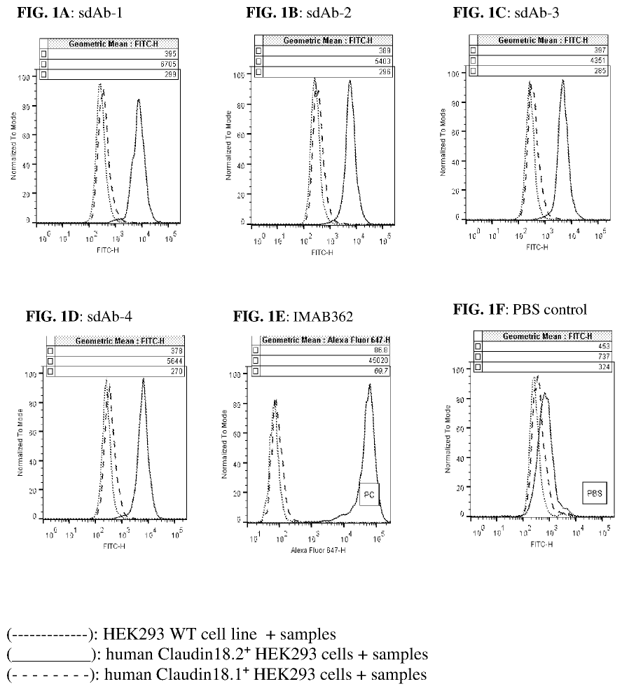

To verify cell surface antigen binding of the sdAbs, the expressed sdAbs in the supernatant were analyzed for their ability to bind to Claudin18.2-expressing HEK293 cells by FACS. Their binding to claudin18.1-expressing HEK293 cells were also evaluated by FACS. HEK293 cells expressing human Claudin18.2 were harvested and incubated with anti-Claudin18.2 sdAbs described herein, followed by FITC-labeled anti-His tag antibody (Genscript, cat #A01620). The samples were then analyzed with flow cytometry. The typical binding figures were shown in FIGS. 1A-1F. The anti-Claudin18.2 mAbs bind specially to human Claudin18.2 expressing cells (solid line), rather than Claudin18.1 proteins expressing cells (dotted line). Negative binding was indicated as when solid line merged with background grey curves which was parental HEK293 cells without engineering. The target binding pattern was illuminated by positive control antibody IMAB362 (Claudiximab) and PBS controls, and IMAB362 binding affinity was detected by fluorophore (iFluor 647)-labeled goat anti-human IgG Fc secondary antibody (Jackson, cat #109-605-098). These sdAbs were sequenced and the unique clones were selected for further characterization. The sequences of identified/tested sdAbs (or clones) are shown in the Sequence Listing appended herein.

Production of sdAb-Fc Fusion Proteins

The anti-Claudin18.2 sdAb-Fc fusion protein constructs were generated by fusion of anti-Claudin18.2 sdAbs with human IgG1 Fc region (with the native IgG1 hinge region). The maxiprep of the constructs were prepared for CHO-3E7 cell transient expression. The expressed anti-Claudin18.2 sdAb-Fc fusion proteins were purified by chromatography through a column containing Protein A agarose resin.

### Example 2

### Characterization of Anti-Claudin18.2 sdAbs In Vitro

### Human Claudin18.1 Cell Binding by FACS Assay

To further investigate antibody binding specificity that only binds to Claudin18.2 but not Claudin18.1 after construction of sdAb-hIgG-Fc fusion proteins, HEK293 cells expressing human Claudin18.1 were incubated with 100 μl anti-Claudin18.2 sdAbs in 3× serial dilutions, starting from the concentration of 300 nM, followed by fluorophore (iFluor 647)-labeled goat anti-human IgG Fc secondary antibody (Jackson, cat #109-605-098) incubation. The samples were then analyzed with flow cytometry. Antibody-antigen binding curves were generated with geometric mean values. Raw data was plotted with GraphPad Prism v6.02 software with four parameters, best-fit values program to analyze the EC₅₀.

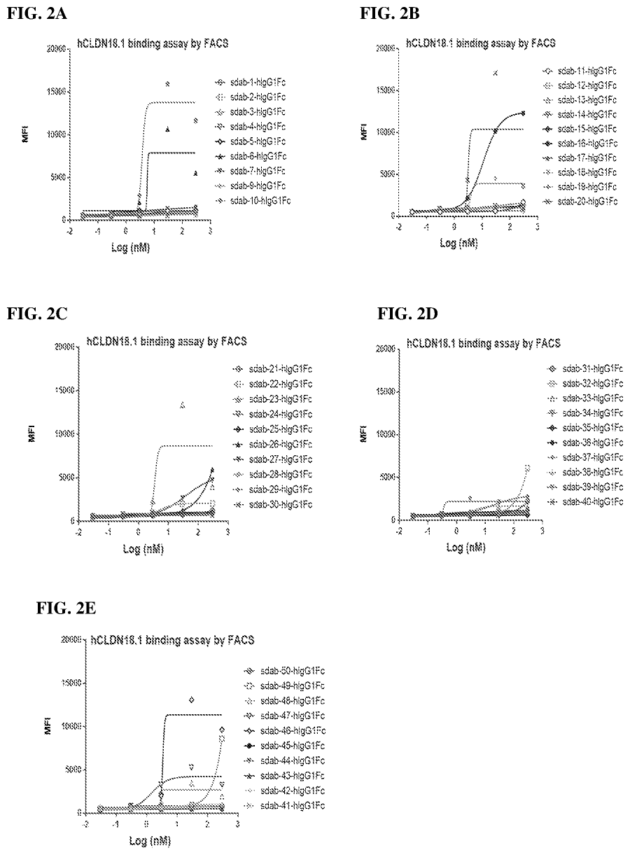

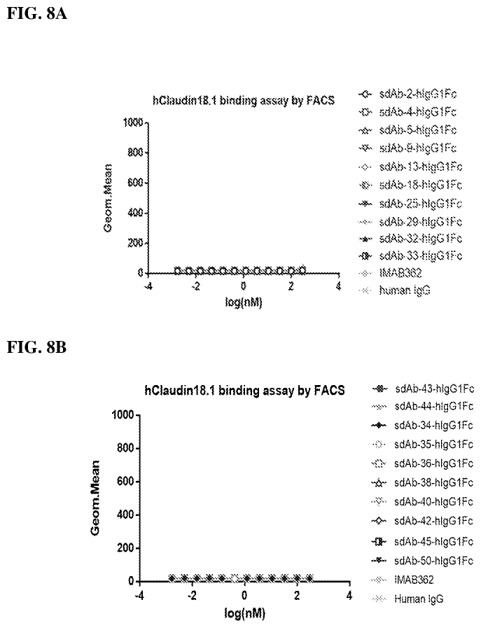

Among these anti-Claudin18.2 sdAb-Fc fusion proteins, as shown in FIGS. 2A-2E, only 6 proteins including sdab-6-hIgG1Fc, sdab-10-hIgG1Fc, sdab-16-hIgG1Fc, sdab-20-hIgG1Fc, sdab-23-hIgG1Fc and sdab-46-hIgG1Fc showed high binding affinity to human Claudin18.1. The other sdAb-Fc fusion proteins showed no or weak binding affinity to human Claudin18.1. To further identify their binding specificity with human Claudin18.1, all of the sdAb-Fc fusion proteins with no or weak binding affinity to human Claudin18.1 were selected for the 2^{nd} round of binding assay by flow cytometry. As shown in FIGS. 8A-8B, none of them binds to human Claudin18.1.

### Human Claudin18.2 Cell Binding by FACS Assay

After analyzing non-specific binding to human Claudin18.1, these candidates with no/weak Claudin 18.1 binding affinity were selected to check their binding affinity to human Claudin18.2. To verify cell surface Claudin18.2 antigen binding of antibody products, HEK293 cells expressing human Claudin18.2 were incubated with 100 μl anti-Claudin18.2 sdAb-Fc fusion proteins in 3× serial dilutions, starting from the concentration of 300 nM, followed by fluorophore (iFluor 647)-labeled goat anti-mouse IgG (H+L) secondary antibodies incubation. The samples were then analyzed with flow cytometry. Antibody-antigen binding curves were generated with geometric mean values. Raw data was plotted with GraphPad Prism v6.02 software with four parameters, best-fit values program to analyze the EC₅₀.

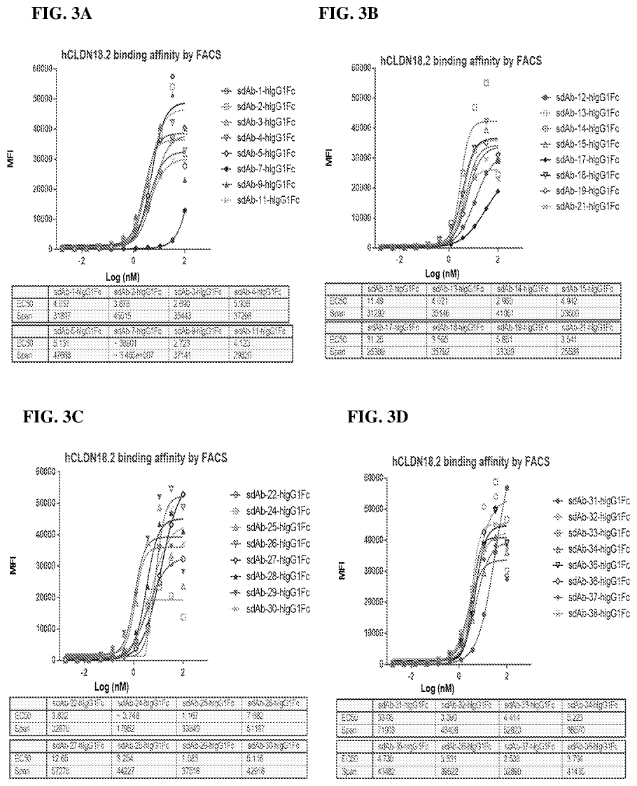

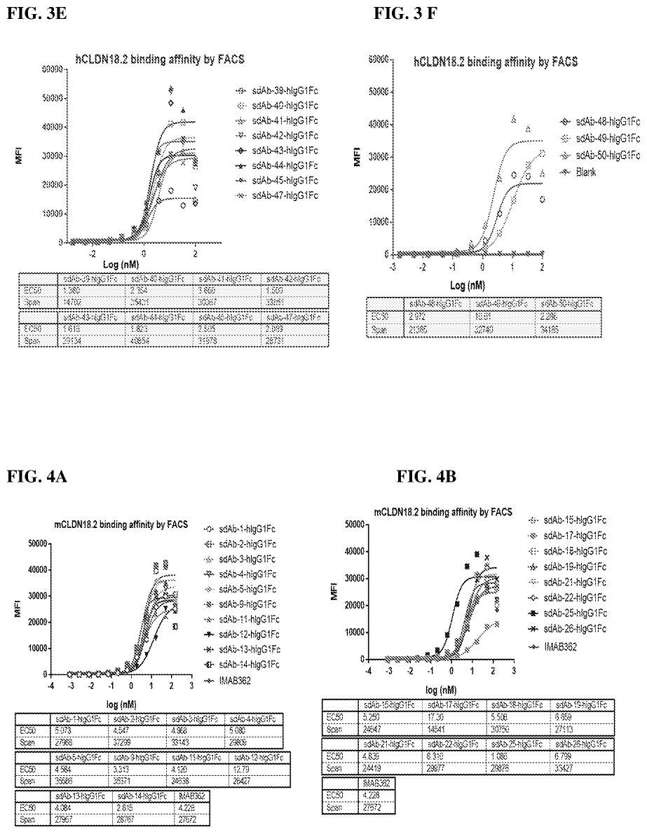

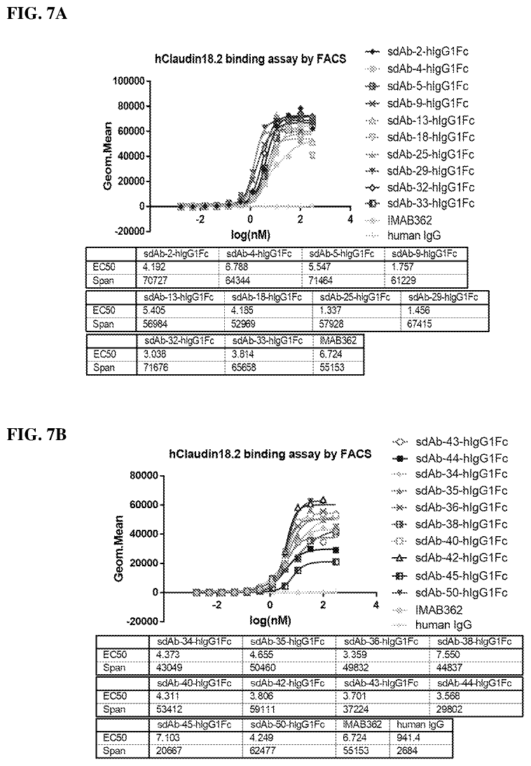

Among these anti-Claudin18.2 sdAb-Fc fusion proteins, as shown in FIGS. 3A-3F, most of anti-Claudin18.2 sdAb-Fc fusion proteins bind to human Claudin18.2 expressing cells with high affinity except sdab-7-hIgG1Fc, sdab-17-hIgG1Fc and sdab-39-hIgG1Fc. To further identify their binding affinity with human Claudin18.2, all of the sdAb-Fc fusion proteins with high binding affinity were selected for the 2^{nd} round of binding assay by flow cytometry. As shown in FIGS. 7A-7B, when compared with reference mAb of IMAB362, all of them were confirmed with similar or higher binding affinity except sdAb-45- hIgG1Fc.

### Mouse Claudin18.2 Cell Binding by FACS Assay

To check binding affinity of these fusion proteins to mouse Claudin18.2, CHO-K1 cells expressing mouse Claudin18.2 were harvested and incubated with 100 μl anti-Claudin18.2 sdAb-Fc fusion proteins in 3× serial dilutions, starting from the concentration of 300 nM, followed by fluorophore (iFluor 647)-labeled goat anti-human IgG Fc secondary antibody (Jackson, cat #109-605-098) incubation. The samples were then analyzed with flow cytometry. Antibody-antigen binding curves were generated with geometric mean values. Raw data was plotted with GraphPad Prism v6.02 software with four parameters, best-fit values program to analyze the EC₅₀.

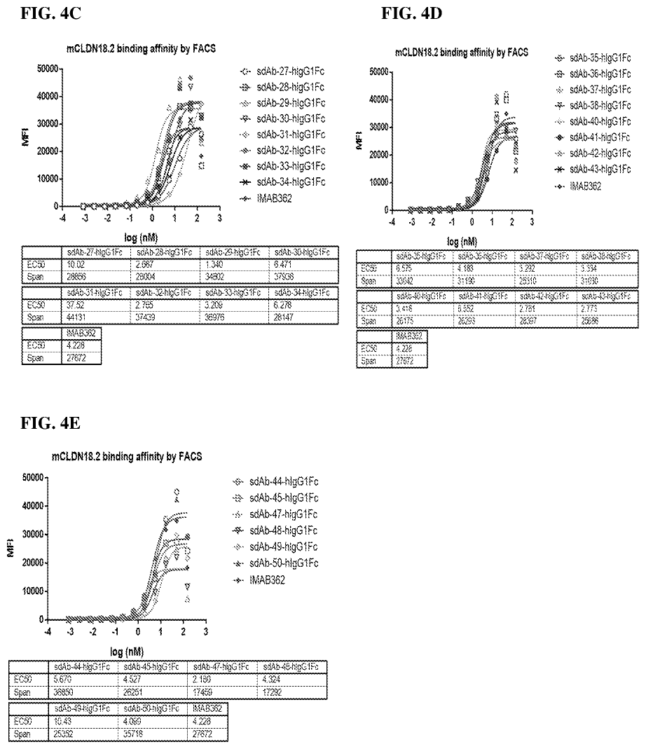

Among the anti-Claudin18.2 sdAb-Fc fusion proteins of the present disclosure, as shown in FIGS. 4A-4E, most of anti-Claudin18.2 sdAb-Fc fusion proteins bind to mouse Claudin18.2 expressing cells with high affinity except sdab-17-hIgG1Fc.

### Anti-Claudin18.2 Antibody Induced Complement Dependent Cytotoxicity (CDC) Assay

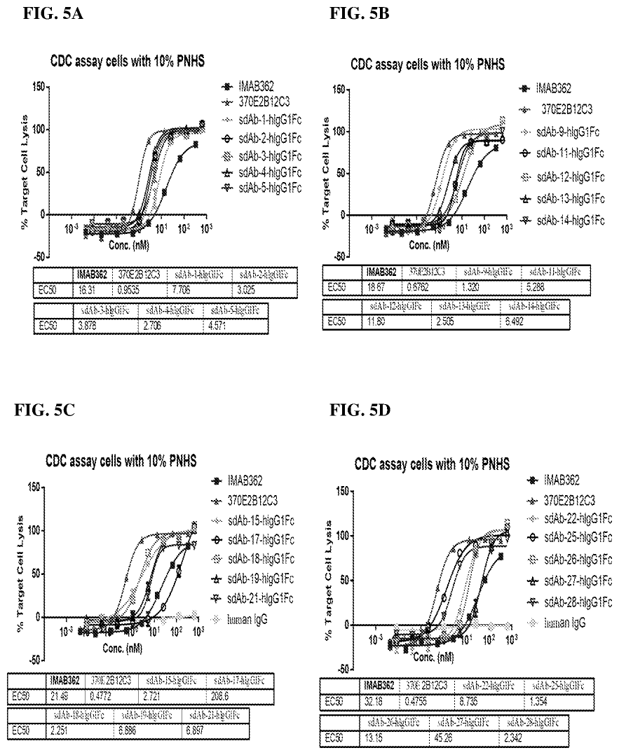

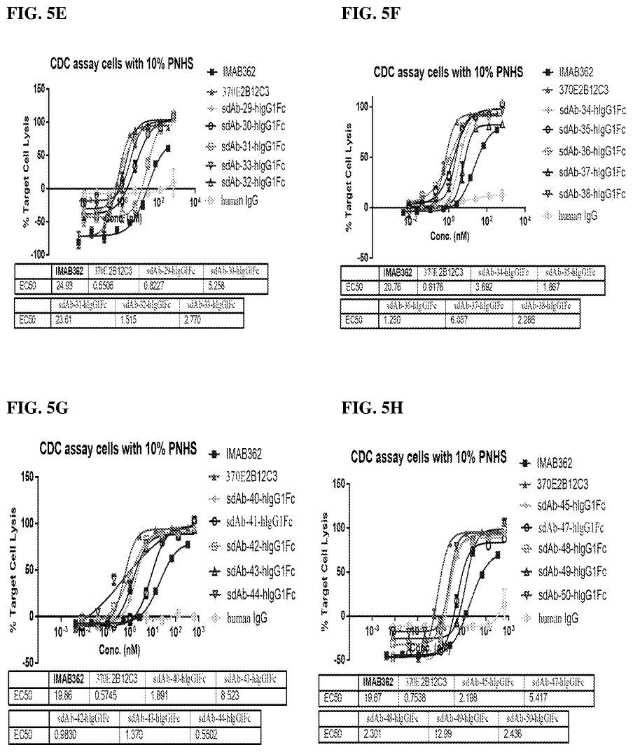

Antibody-dependent cellular cytotoxicity (ADCC) and complement-dependent cytotoxicity (CDC) effect are the major mechanisms of action for anti-human Claudin18.2 therapeutic antibodies against human gastric or gastro-oesophageal carcinoma. The candidate anti-Claudin18.2 sdAb-Fc fusion proteins were functionally assessed using a CDC assay. Briefly, the target cell line, CHO-K1 overexpressing human Claudin 18.2, was cultured and harvested, and it was seeded in 96-well plate. Antibody samples were added to the plate accordingly and the plate was incubated at 37° C./5% CO₂ for 30 min. Purified normal human serum was then added to the plate and the plate was incubated further for 4 hours. The plate was taken out of the incubator and the supernatant was collected and analyzed with Cell Titer-Glo® assay kit (Promega, cat #G7573). The luminescence data was captured by PheraStar (AMG) for cell viability analysis. Two positive controls, including IMAB362 (Zolbetuximab) and 370E2B12C3 (disclosed in WO202013567A1) was used for comparison. The CDC assay results in term of % target cell lysis versus candidate antibody concentration are shown in FIGS. 5A-5H. All of sdAb-Fc fusion proteins, except sdab-17-hIgG1Fc and sdab-27-hIgG1Fc, showed better CDC activity than reference mAb of IMAB362. When compared with 370E2B12C3, many sdAb-Fc fusion proteins showed comparable activity including sdab-2-hIgG1Fc, sdab-4-hIgG1Fc, sdab-9-hIgG1Fc, sdab-25-hIgG1Fc, sdab-29-hIgG1Fc, sdab-32-hIgG1Fc, sdab-33-hIgG1Fc, sdab-35-hIgG1Fc, sdab-36-hIgG1Fc, sdab-38-hIgG1Fc, sdab-40-hIgG1Fc, sdab-42-hIgG1Fc, sdab-43-hIgG1Fc, sdab-44-hIgG1Fc, sdab-45-hIgG1Fc, sdab-48-hIgG1Fc and sdab-50-hIgG1Fc.

### Anti-Claudin 18.2 Antibody Induced ADCC Assay

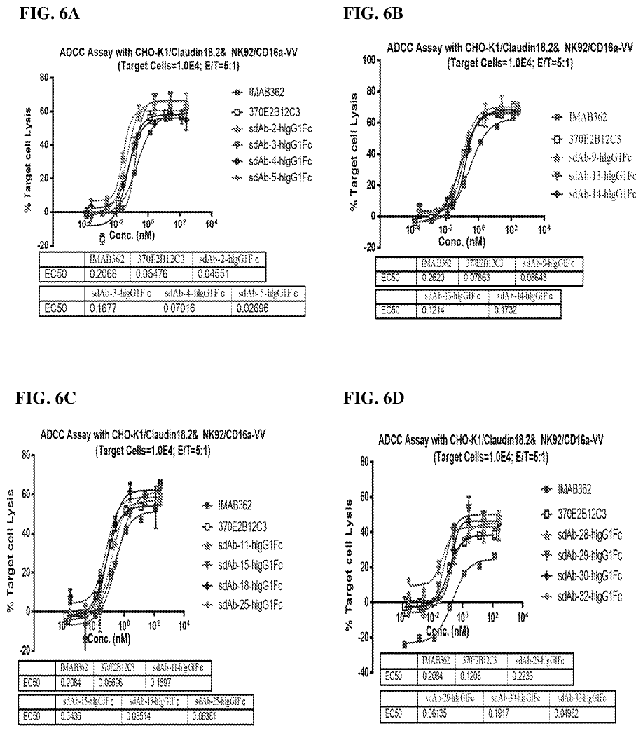

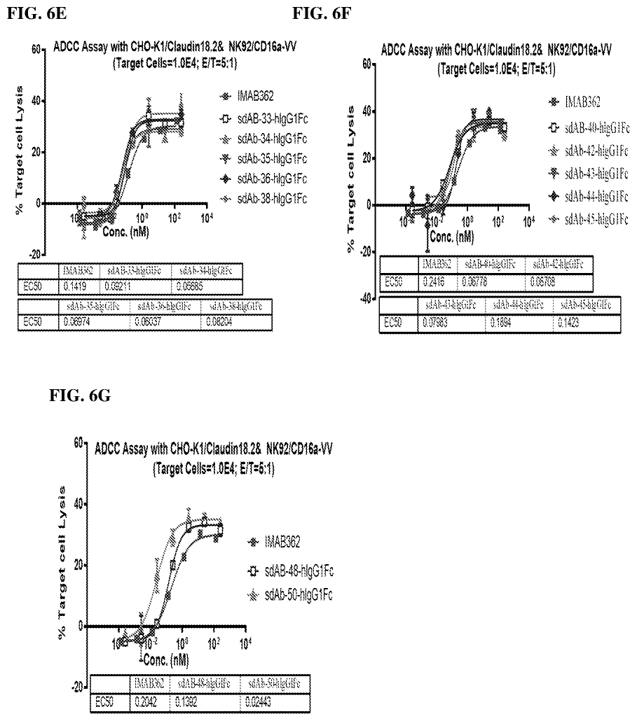

The ADCC effect of anti-Claudin18.2 sdab-Fc fusion proteins were compared. The ADCC assay results in term of % target cell lysis versus candidate antibody concentration are shown in FIGS. 6A-6G. For the assay procedure, the target cell line, CHO-K1-overexpressing human Claudin18.2, was cultured, harvested and seeded into 96 well plates. The samples or positive control, which was in-house synthesized with the identical amino acid sequence of IMAB362 (Zolbetuximab), were added to the plate and the plate was incubated at 37° C./5% CO₂ for 30 min. NK92 cells were used as the effector cells and added to the plates and incubated at the same condition for 6 hours. The assay plate was taken out and short centrifuged. The supernatant was collected and transferred to a new plate for LDH activity assay as per manufacturer's instruction (Roche). The absorbance data were captured by FlexStation 3 and analyzed by GraphPad Prism 6.0. Two positive controls, including IMAB362 (Zolbetuximab) and 370E2B12C3 (WO202013567A1) was used for comparison. The ADCC assay results in term of % target cell lysis versus candidate antibody concentration are shown in FIGS. 6A-6G. All of sdAb-Fc fusion proteins showed better ADCC activity than reference mAb of IMAB362.

### Example 3

### Antibody Humanization and Analysis

### Antibody Humanization and Characterization

Based on antibody variable domain sequences, the CDRs and FRs were analyzed and homology modeling was performed to obtain the modeled structure of the parental llama antibodies of sdab-09, sdab-25, sdab-29, sdab-32, sdab-42 and sdab-50. Antibody humanization was performed through the the following methods: 1) Calculate the solvent accessible surface area of framework residues. 2) Based on the result, identify framework residues that are buried (i.e. with solvent accessible surface area of <15%). 3) Select human acceptors for sdAbs that share high sequences identity to the sdAb counterparts. 4) Directly graft the CDRs of the llama sdAb to the human acceptor frameworks to obtain the sequence of the grafted antibody without any back mutation. 5) Analyze post translational modifications and chemical degradation in grafted sequence including deamidation, isomerization oxidation and glycosylation etc. through developability assessment. 6) Identify PTM hotspots like N-glycosylation sites, unusual proline residues, deamidation site, isomerization site, oxidation site and unpaired cysteine residues etc. that may affect the binding activity and manufacturability of the grafted antibody.

For sdAb-Fc fusion protein construction, anti-Claudin18.2 humanized sdAb was fused to the N-terminus of human IgG1 Fc region. These humanized antibody fusion proteins were expressed in CHO-3E7 cells. Twenty four hours later, the expression/secretion was boosted with Tryptone N-1 supplement. After 6 days of shaking culture in 37° C. and 5% CO₂, supernatants were collected and the humanized antibodies were purified with Protein-A beads as described above. The humanized antibodies were kept in PBS for analysis.

### Anti-Claudin18.2 Antibody Induced ADCC Assay

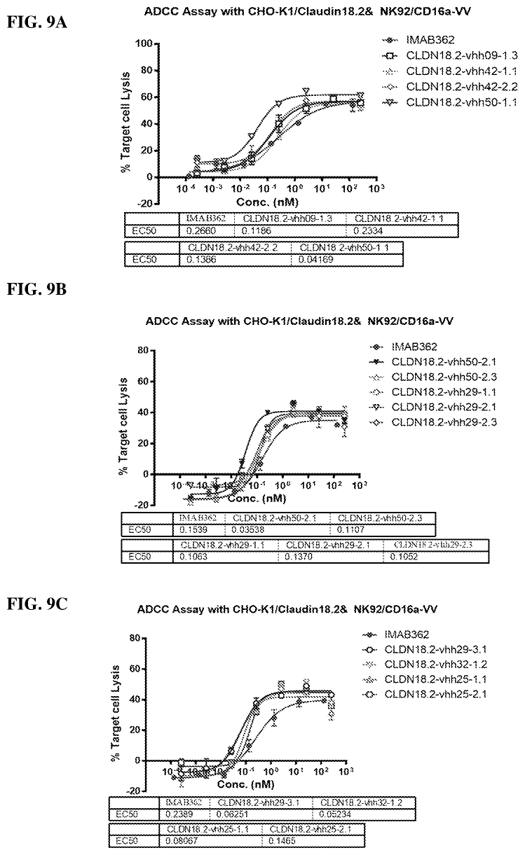

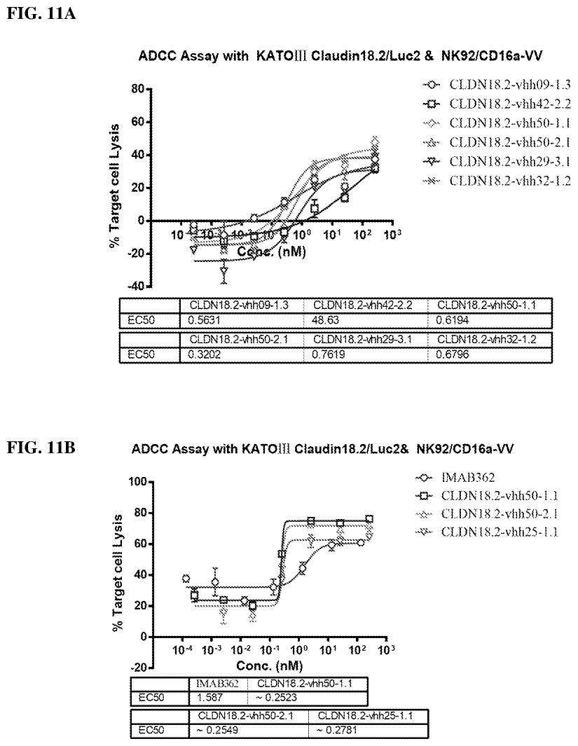

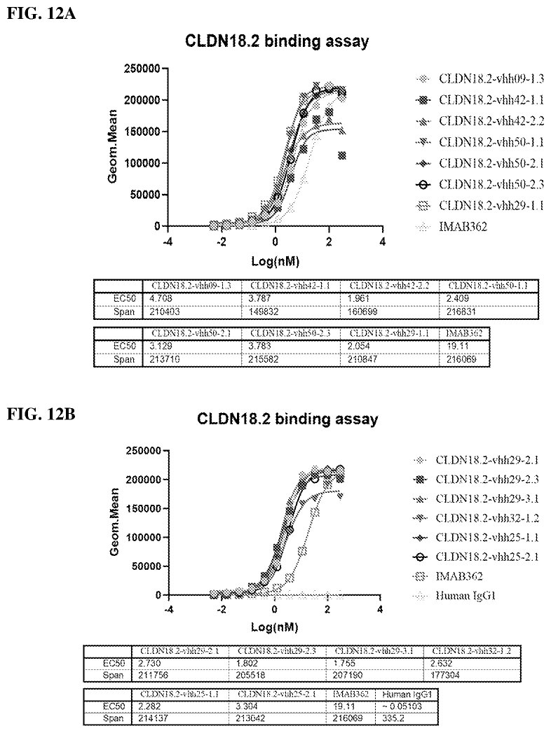

The ADCC effect of all the humanized antibodies were compared. For the assay procedure, the target cell line, CHO-K1 or KATO III overexpressing human Claudin18.2, was cultured, harvested and seeded into 96 well plates with 1×10⁴ cells. The humanized antibody samples or positive control, which was in-house synthesized with the identical amino acid sequence of IMAB362 (Zolbetuximab), were added to the plate and the plate was incubated at 37° C./5% CO₂ for 30 min. NK92 cells were used as the effector cells and added to the plates and incubated at the same condition for 6 hours. The assay plate was taken out and short centrifuged. The supernatant was collected and transferred to a new plate for LDH activity assay as per manufacturer's instruction (Roche). The absorbance data were captured by FlexStation 3 and analyzed by GraphPad Prism 6.0. When the target cell line of CHO-K1 overexpressing human Claudin18.2 was utilized for ADCC bioassay, as shown in FIGS. 9A-9C, the humanized sdAb leads of CLDN18.2-vhh50-1.1, CLDN18.2-vhh50-2.1, CLDN18.2-vhh29-3.1, CLDN18.2-vhh32-1.2 and CLDN18.2-vhh25-1.1 exhibited much better ADCC activity than reference antibody of IMAB362. Also, the humanized sdAb leads of CLDN18.2-vhh09-1.3, CLDN18.2-vhh42-1.1, CLDN18.2-vhh42-2.2, CLDN18.2-vhh29- 1.1, CLDN18.2-vhh29-2.1, CLDN18.2-vhh29-2.3, CLDN18.2-vhh50-2.3 and CLDN18.2-vhh25-2.1 showed a little higher ADCC activity then reference antibody of IMAB362. When the target cell line of KATO III overexpressing human Claudin18.2 was utilized for ADCC bioassay, as shown in FIGS. 11A-11B, the humanized sdAb leads of CLDN18.2-vhh50-1.1, CLDN18.2-vhh50-2.1, CLDN18.2-vhh29-3.1, CLDN18.2-vhh32-1.2, CLDN18.2-vhh09-1.3 and CLDN18.2-vhh25-1.1 exhibited much better ADCC activity than reference antibody of IMAB362.

### Anti-Claudin18.2 Antibody Induced Complement Dependent Cytotoxicity (CDC) Assay

The humanized candidates of anti-Claudin18.2 sdAb-Fc fusion proteins were functionally assessed using a CDC assay. Briefly, the target cell line, 5000 of CHO-K1 cells overexpressing human Claudin 18.2, was cultured and harvested, and it was seeded in 96-well plate. Antibody samples in 5× serial dilutions, starting from the concentration of 50 μg/ml, were added to the plate accordingly and the plate was incubated at 37° C./5% CO₂ for 30 min. Purified normal human serum was then added to the plate and the plate was incubated further for 4 hours. The plate was taken out of the incubator and the supernatant was collected and analyzed with Cell Titer-Glo® assay kit (Promega, cat #G7573). The luminescence data was captured by PheraStar (AMG) for cell viability analysis. IMAB362 (Zolbetuximab) was used as the reference antibody for comparison. The CDC assay result was shown in FIG. 10. All of the humanized sdAb-Fc fusion proteins showed much higher CDC activity than reference mAb of IMAB362.

### Human Claudin18.2 Expressing Cell Binding Assay by FACS

To verify cell surface Claudin18.2 antigen binding of antibody products, CHO-K1 cells expressing human Claudin18.2 were incubated with 100 μl humanized anti-Claudin18.2 sdAb-Fc fusion proteins in 3× serial dilutions, starting from the concentration of 300 nM, followed by fluorophore (iFluor 647)-labeled goat anti-mouse IgG (H+L) secondary antibodies incubation. The samples were then analyzed with flow cytometry. Antibody-antigen binding curves were generated with geometric mean values. Raw data was plotted with GraphPad Prism v6.02 software with four parameters, best-fit values program to analyze the EC₅₀.

As shown in FIGS. 12A-12B, all of humanized sdAb leads exhibited higher binding affinity than reference antibody of IMAB362.

### SEQUENCE LISTING

The full amino acid sequences of the clones in the Examples are set forth below. The underlined amino acid sequences signify CDR1, CDR2, CDR3, respectively, of a given clone.

>sdab-1 (SEQ ID NO: 1)
QVKLEESGGGLAQAGGSLRLSCAASGRILNIAVMAWYRQTPGRQRELVAAITFGGSTDY
ADAVKGRFTISRDSAKNTVYLQMNDLKSEDTAVYYCNADVVTGLMRRDVYWGQGTQ
VTVSS
>sdab-2 (SEQ ID NO: 2)
QVKLEESGGGLVQAGGSLRLSCAASVEIFMMGWYRQAPGKQRELVATISSSGRTNYAD
SVKGRFTISGDNSKNTVYLQMNSLKPEDTAVYYCYCHYWRDYWGRGTQVTVSS
>sdab-3 (SEQ ID NO: 3)
QVKLEESGGGLVQAGGSLRLSCAASGSIYRINNMAWYRQAPGKQRELVAEITSSDNTY
YADSVKGRFTISRDNAKNTVYLQMNSLKPEDTAVYSCNAFIIPSVGVESSRTYWGRGTQ
VTVSS
>sdab-4 (SEQ ID NO: 4)
QVQLVESGGGLVQPGGSLRLSCAASGSIFQINTMGWYRQAPGKQRELVAAITTGGSTKY
SDSVKGRFTISRDNAKNTVYLQMNSLKPEDTAVYYCNADLKTNSNVFTWYEYWGQGT
QVTVSS
>sdab-5 (SEQ ID NO: 5)
DVQLVESGGGLVQAGGSLRLSCAASVGIFMGGWYRQAPGKQRELVATISSSGRTNYAD
SVKGRFTISGDNSMNTVYLQMNSLKPEDTAVYYCYAHYWRDYWGRGTQVTVSS
>sdab-6 (SEQ ID NO: 6)
AVQLVDSGGGLVQAGGSLRLSCAPSGKIFSIHNMGWYRQAPGKQRELVASTTNDGTTT
YADSVKGRFIISRDIAKNTVYLQMQSLKPEDTAVYYCNAFIVTGEMASGDYWGQGTQV
TVSS
>sdab-7 (SEQ ID NO: 7)
DVQLVESGGGLVQPGGSLRLSCAASGFTFSTYAMSWVRQAPGKGLEWVSSTSSSGRTT
SYSDSVKGRFTISRDNAKSTLYLQMNSLKPEDTAVYYCAKAGAYSGTDYYTLVRYYDY
WGQGTPVTVSS
>sdab-8 (SEQ ID NO: 8)
DVQLVESGGGLVQPGGSLRLSCAASGFTFDDYGMSWVRQAPGKGLEWVSGISWRGGS
TYYAESIKGRFTASRDNAKDTVYLQMNSLKPEDTAVYYCVRGSYYYNSRGYDYWGQG
TPVTVSS
>sdab-9 (SEQ ID NO: 9)
DVQLVESGGGLVQAGGSLTLSCAASRSDLFISIMGWYRQAPGKQRELVADISRGGKTNY
ADAVKARATISRDNAKNTMYLQMNNVKPEDTAVYYCYAQISNPFWGDYWGRGTQVT
VSS
>sdab-10 (SEQ ID NO: 10)
QVKLEESGGGFVQAGGSLRLSCSASGSIFGITGMAWYRQAPGKERELIAAIGSGSAGNT
KYAESVKGRFTIFGDYAKKTLYLQMNSLKPEDTGVYYCNAGLMTSQSYYSKPYWGLG
TQVTVSA
>sdab-11 (SEQ ID NO: 11)
EVQLVESGGGLVQAGGSLRLSCVASGTILNIPLMAWFRQTPGKQRELVAGITTGGTTNY
ADSAKGRFTISRDNAKKTIYLQMDSLKPEDTAVYYCNADVVTGLMRRNVYWGQGTQV
TVSS
>sdab-12 (SEQ ID NO: 12)
QVKLEESGGGLVQAGGSLTLSCAASRSIFSIDVMAWYRQAPGKQRELVARIWADGSTT
YADSMKGRFAISRDTAKNTVYLQMNSLKPEDTAVYYCNAVGTIADGAFRPYNNWGQG
TQVTVSS
>sdab-13 (SEQ ID NO: 13)
AVQLVESGGGLAQAGGSLRLSCAASGRILNMASMAWFRQTPGKQRESVALITFGGTTN
YADSVKGRFTISRDNAKNTVYLQMNDLKSEDTAVYYCNADVVTGLMRRDVYWGQGT
QVIVSS
>sdab-14 (SEQ ID NO: 14)
QVKLEESGGGLVQAGGSLRLSCAASGSIYRINNMAWYRQAPGKQRELVAEITSSGNTD
YADSVKGRFTISRDNAKNTVYLQMNSLKPEDTAVYSCNAFIIPSVGVFTSRTYWGRGTQ
VTVSS
>sdab-15 (SEQ ID NO: 15)
QVKLEESGGGLVQAGGSLRLSCAASGSILNINSMSWYRQAPGKQRELVATITVGGSTNY
ADSVKGRFTISRYNANNMMYLQMNSLKPDDTGVYSCNARIVSGFLRGPDDYWGQGTQ
VTVSS
>sdab-16 (SEQ ID NO: 16)
QVQLVESGGQSAQAGGSLRLSCTASGSIFGISGMGWYRQAPGKERELIAAIGSGGDGDT
KYAESVKGRFTISGDYAKKTLYLQMNSLKPEDSGVYYCNAGFMTSRNYYNGDYWGQG
TQVTVAA
>sdab-17 (SEQ ID NO: 17)
EVQLVESGGGLVQAGGSLRLSCVWSGSFDDIGTMGWYRQAPGKQRELVAIATNGAIQN
YAESVKGRFTVSRDNAKNTLYLQMNNLKPEDTAVYYCKMDSQFGPWLRQYEYWGQG
TQVTVSP
>sdab-18 (SEQ ID NO: 18)
AVQLVESGGGLVQAGGSLRLSCAASGSIFMINAMGWYRQAPGKQRELVADISRGGSTY
YADSVKGRFTISRDNDKNTVSLQMNSLKPEDTAAYYCHAHDVPPFVPGGKSYWGQGT
QVTVSS
>sdab-19 (SEQ ID NO: 19)
QVKLEESGGGLVQAGGSLRLSCAASVSIDGILAMGWYRQAPGKQRELVATIHSRGNTN
YADSVKGRFTVSRENNKNTMYLQMNSLKPEDTAVYYCYVHFWIDYWGQGTQVTVSS
>sdab-20 (SEQ ID NO: 20)
QVKLEESGGGLVQAGGSLRLSCVASGSFLHIAVMGWYRQAPGKQRELVATITRGGATH
YADSTKGRFSISSDNAKNAVALQMNSLQPDDTAVYFCNVRALIKVFHPPNDYWGQGTQ
VTVSS
>sdab-21 (SEQ ID NO: 21)
QVQLVESGGGLVQAGGSLRLSCAASGRILNLNDMGWYRQTPGKQRELVASIFSSGTTN
YADSAKGRFTISRDNAKKMIYLQMNSLKPEDTAVYYCNADVVTGLLQRNVYWGQGTQ
VTVSS
>sdab-22 (SEQ ID NO: 22)
AVQLVESGGGLVQAGGSLRLSCVASGSWFRIQTMGWYRQTPGKQRELVADITTGGGST
YYVDSVKGRFTISRDNAKNTVYLQMINLKPEDTAAYYCQANVLEALFRPNLEVWGQGT
LVTVSS
>sdab-23 (SEQ ID NO: 23)
AVQLVDSGGGLVQAGGSLRLSCAASGIWLHMVTMGWYRQAPGKQRELVAIITSGGSA
DYADSVKGRFAISRDNTKNTVTLQMNSLKPDDTAVYFCNARVVADFQKVLDVWGQGT
LVTVSS
>sdab-24 (SEQ ID NO: 24)
QVKLEESGGGLVEAGGSLRLSCVASGSILNLMTMGWYRQAPGKQRELVATATPGGTTN
YADSVKGRFTISRDNAKNTVYLQMNTLRPGDTAVYYCNADVVYGLARQGNYWGPGT
QVTVSS
>sdab-25 (SEQ ID NO: 25)
QVKLEESGGGLVQAGGSLRLSCVASGSIYRINNMAWYRQAPGKQRELVAESTSGGNFIY
ADSVKGRFAISRDNAKNTVYLQMNSLQPEDTAVYSCNAFIIPSVGVFASRTYWGRGTQV
TVSS
>sdab-26 (SEQ ID NO: 26)
DVQLVESGGGPVQAGGSLRLSCVASGSIYRINEMAWYRQAPGKQRELVAVAPNSVNPN
YADSVKGRFTISRGNAKNTVYLQMNSLKPEDTAVYSCNAFIIPSVGVFTSRVYWGRGTQ
VTVSS
>sdab-27 (SEQ ID NO: 27)
AVQLVDSGGGLVQAGGSLRLSCAVSGSFLNIVTMGWYRQAPGKQRELVATTTRGGSTN
YADSVKGRFTIARDNAKNTVYLHMDALKPEDTAVYYCNAEIIPGFLQQLTLYWGQGTQ
VTVSA
>sdab-28 (SEQ ID NO: 28)
QVKLEESGGGLVQAGGSLRLSCAASGSSNADIRFMGWYRQPAGNQRNNQREPVAMVD
TTGRINYADSVKGRFTISRDNAKNTVSLQMNSLKPEDTALYYCTAVLWPRGAISAMTV
WGQGTQVTVSS
>sdab-29 (SEQ ID NO: 29)
QVKLEESGGGLVQAGGSLRLSCAASGSIYRINNMAWYRQIPGKQRELVAEITSSGNTLY
ADSVKGRFTISMDNAKNTAYLQMNSLKPEDTAVYSCNAFIIPSVGVFISRTFWGRGTQV
TVSS
>sdab-30 (SEQ ID NO: 30)
QVQLVESGGGLVQAGGSLRLSCAASGSIYRINNMAWYRQAPGKQRELVAEITSSGNTL
YADSVKGRFTISSDGARNTVYLQMNSLKPEDTAVYSCNAFIIPSVGVFTSRAYWGRGTQ
VTVSS
>sdab-31 (SEQ ID NO: 31)
AVQLVESGGDLVQAGGSLRLSCAASGSIFGFVVMGWYRQAPGNQRELVAMLTSSGTTN
YADSVKGRFTLFRENNKNTVHLQMNSLKPEDTGVYYCRAVIRSDSSWRADANEYWGQ
GTHVTVSS
>sdab-32 (SEQ ID NO: 32)
QVKLEESGGGLVQAGGSLRLSCAIPGSIVGSTYMGWFRQAPGKQRELVARIAGGGQINY
ADSVKGRFTISRDNAKNSLYLQMNSLTPEDTAVYYCYVHYWMDYWGQGTQVTVSS
>sdab-33 (SEQ ID NO: 33)
QVKLEESGGGLVQAGGSLRLSCAASGSIYRINNMAWYRQAPGKQRELVAEITNSGNTL
YTDSVKGRFTISMDGAKNAVYLQMNSLKPEDTAVYSCNAFIIPSVGVFTSRAYWGRGT
QVTVSS
>sdab-34 (SEQ ID NO: 34)
QVKLEESGGGLVQTGGSLSLSCVASGSGISINTMGWYRQGPGKQRELVAIVGKYGETNY
ADSVKGRFTISRDDAKKTVYLRMNSLKPEDTAVYYCNADVLAAGWWSGRVLWGQGT
QVTVSS
>sdab-35 (SEQ ID NO: 35)
AVQLVESGGGLVQAGGSLRLSCVASGSIFQINTMGWYRQAPGKQREWVTGITTGGTTR
YADSVKGRFTISRDNAKNTVYLQMNSLKPEDTAVYYCNSDLKTTSNVFQWYEYWGQG
TQVTVSA
>sdab-36 (SEQ ID NO: 36)
QVKLEESGGGLVQAGGSLRLSCAISGSIFSNTYMGWYRQAPGRQRELVARISGGGSVHY
LDSVKGRFTISRDNAKNTLNLQMNSLTPEDTAIYYCYAHYWMDYWGQGTQVTVSS
>sdab-37 (SEQ ID NO: 37)
QVKLEESGGGLVQAGGSLRLSCAVSGNFLNIVTMGWYRQAPGKQRELVATTTRGGSTS
YSDSVKGRFTISRDNAKSTVYLQMNSLKPEDTAVYYCNADIIPGFLQQLTVHWGQGTR
VTVSS
>sdab-38 (SEQ ID NO: 38)
EVQLVESGGGLVQAGGSLRLSCVASGSIFHIPIMSWYRRAPGKQRELVAGISTGGSTNYA
DSVKGRFTISRDNAKNTVYLQMNSLKPDDTAVYYCNSLIKEEAWGTFGDYWGQGTQV
TVSS
>sdab-39 (SEQ ID NO: 39)
QVKLEESGGGLAEAGGSLRLSCVASGSIFMIVTMGWYRQAPGKQRELVASITRGGSTNY
ADFVKGRFTISRDNAKNTVYLQMNSLKPEDTAVYFCNADDVPPLTLTRVIYWGQGTQV
TVSS
>sdab-40 (SEQ ID NO: 40)
QVQLVESGGGLVQAGGSLRVSCAASGRTSSIYDMAWYRQAPGNEREFVARIVWRSGGS
TSYADSVKGRFTISRNTAKNTVYLQMNSLKPEDTAMYYCRAFEFPLQIGGQNVYYWGQ
GTQVTVSS
>sdab-41 (SEQ ID NO: 41)
QVQLVESGGDLVQAGGSLRLSCAASGSPFGFVVMGWYRQAPGKQRDMVATLTSSGAT
TYADSVKGRFTLSRENAKNTIHLQMNSLKPEDTAVYYCRAVLRSGDNWMAAANEYW
GQGTHVTVSS
>sdab-42 (SEQ ID NO: 42)
EVQLVESGGGLVQAGGSLRLACAASGRAIGTYDTNWYRQAPGQQREFVAFINYRGGGA
TYADSVKDRFTISRDNAANTVYLQMNSLKPEDTAIYLCNAHERFPMGRPPYDYWGQGT
QVTVTS
>sdab-43 (SEQ ID NO: 43)
EVQLVESGGGLVQAGGSLRLSCEASGPARNNYYMGWFRQAPGKERELVARINLSGSSI
YYADSVKGRFTISRDNAKNTMSLQMNSLKPGDAARYYCHARDIVPGAQDYWGQGTQV
TVSA
>sdab-44 (SEQ ID NO: 44)
EVQLVESGGGLVQAGGSLRLSCAASGSIYRINTVAWYRQAPGKQRELVAEITSSGNRDY
ADSVKGRFTISRDNAKNTVYLQMNSLKPEDTAVYSCNAFIIPSVGLFTSRTYWGRGTQV
TVSS
>sdab-45 (SEQ ID NO: 45)
QVKLEESGGGLVQAGGSLRLSCAASGSISGINYMAWYRQAPGKQRDLVARAWTTGPPT
YAESVKGRFTISRDNAKNTVYLQMNNLKPEDTGVYYCTANTLTSGYWGQGIQVTVSS
>sdab-46 (SEQ ID NO: 46)
AVQLVDSGGGLVQAGGSLRLSCAASPTIFVTDVMAWYRQAPGNQRELVATLLGRSTTN
YADSVKGRFTISRDNAKNTVYLQMNTLKPEDTAVYYCNLHRNLENKIDNYWGQGTQV
TVSS
>sdab-47 (SEQ ID NO: 47)
QVKLEESGGGLVQAGGSLRLSCAVSGNFLNIVTMGWYRQAPGKQRELVATTTRGGTTN
YSDSVKGRFTISRDNAKNTVYLQMNSLKPEDTAVYYCNADIIPGFLQQLTVHWGQGAQ
VTVSS
>sdab-48 (SEQ ID NO: 48)
QVKLEESGGGLVQAGGSLGLSCAASGTILHVMVMGWYRQAPGKQRELVATQTIGGTT
NYADSVKGRFTISRDNAKNTVYLQMNTLTPGDTAVYYCNADVVYGLARSGNYWGPGT
QVTVSS
>sdab-49 (SEQ ID NO: 49)
QVQLVESGGDLVQAGGSLRLSCAASGSPFGFVVMGWHRQAPGKQRELVAMLTSSGTT
NYADSVKGRFTLSRENTKNTVHLQMNKLKPEDTAVYYCRAVLRSGSDWRAAANEYW
GQGTHVTVSS
>sdab-50 (SEQ ID NO: 50)
QVKLEESGGGLVQAGGSLRLSCAASGSIYRINAMAWYRQAPGKQREYVAEITSTGNTD
YADSVKGRFTISRDNAENTVYLQMNSLKPEDTAVYSCNAFIIPSVGMFTSRTYWGRGTQ
VTVSS
Human IgG1 Fc region: (SEQ ID NO: 51)
EPKSGDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
The following protein sequences are humanized sdAb sequences:
>CLDN18.2-vhh09-1.3 (SEQ ID NO: 202)
EVQLVESGGGLVQPGGSLRLSCAASRSDLFISIMGWYRQAPGKGREWVSDISRGGKTNY
ADAVKARFTISRDNSKNTLYLQMNSLRAEDTAVYYCAAQISNPFWGDYWGQGTLVTV
SS
>CLDN18.2-vhh42-1.1 (SEQ ID NO: 203)
QVQLVESGGGVVQPGGSLRLSCAASGRAIGTYDTNWYRQAPGKGREFVAFINYRGGGA
TYADSVKDRFTISRDNSKNTLYLQMNSLRAEDTAVYYCAAHERFPMGRPPYDYWGQG
TLVTVSS
>CLDN18.2-vhh42-2.2 (SEQ ID NO: 204)
QVQLVESGGGVVQPGGSLRLSCAASGFTFSTYDTNWYRQAPGKGREFVAFINYRGGGA
TYADSVKDRFTISRDNSKNTLYLQMNSLRAEDTAVYYCAAHERFPMGRPPYDYWGQG
TLVTVSS
>CLDN18.2-vhh50-1.1 & CLDN18.2-vhh50-2.1 (SEQ ID NO: 205) they
share the same protein sequence
EVQLVESGGGLVQPGGSLRLSCAASGSIYRINAMAWYRQAPGKGREYVSEITSTGNTDY
ADSVKGRFTISRDNSKNTLYLQMNSLRAEDTAVYYCAAFIIPSVGMFTSRTYWGQGTLV
TVSS
>CLDN18.2-vhh50-2.3 (SEQ ID NO: 206)
EVQLVESGGGLVQPGGSLRLSCAASGSIYRINAMAWYRQAPGKGREWVSEITSTGNTD
YADSVKGRFTISRDNSKNTLYLQMNSLRAEDTAVYYCAAFIIPSVGMFTSRTYWGQGTL
VTVSS
>CLDN18.2-vhh29-1.1 (SEQ ID NO: 207)
EVQLVESGGGLIQPGGSLRLSCAASGSIYRINNMAWYRQAPGKGRELVSEITSSGNTLYA
DSVKGRFTISRDNSKNTLYLQMNSLRAEDTAVYYCAAFIIPSVGVFISRTFWGQGTTVTV
SS
>CLDN18.2-vhh29-2.1 (SEQ ID NO: 208)
QVQLVESGGGLVKPGGSLRLSCAASGSIYRINNMAWYRQAPGKGRELVSEITSSGNTLY
ADSVKGRFTISRDNAKNSLYLQMNSLRAEDTAVYYCAAFIIPSVGVFISRTFWGQGTLVT
VSS
>CLDN18.2-vhh29-2.3 (SEQ ID NO: 209)
QVQLVESGGGLVKPGGSLRLSCAASGSIYRINNMAWYRQAPGKGREWVSEITSSGNTL
YADSVKGRFTISRDNAKNSLYLQMNSLRAEDTAVYYCAAFIIPSVGVFISRTFWGQGTL
VTVSS
>CLDN18.2-vhh29-3.1 (SEQ ID NO: 210)
EVQLVESGGGLVQPGGSLRLSCAASGSIYRINNMAWYRQAPGKGRELVSEITSSGNTLY
ADSVKGRFTISRDNSKNTLYLQMNSLRAEDTAVYYCAAFIIPSVGVFISRTFWGQGTLVT
VSS
>CLDN18.2-vhh32-1.2 (SEQ ID NO: 211)
EVQLVESGGGLVQPGGSLRLSCAASGFTVSSTYMGWFRQAPGKGRELVSRIAGGGQIN
YADSVKGRFTISRDNSKNTLYLQMNSLRAEDTAVYYCAAHYWMDYWGQGTLVTVSS
>CLDN18.2-vhh25-1.1 (SEQ ID NO: 212)
EVQLVESGGGLVQPGGSLRLSCAASGSIYRINNMAWYRQAPGKGRELVSESTSGGNFIY
ADSVKGRFTISRDNSKNTLYLQMNSLRAEDTAVYYCAAFIIPSVGVFASRTYWGQGTLV
TVSS
>CLDN18.2-vhh25-2.1 (SEQ ID NO: 213)
EVQLVESGGGLVQPGGSLRLSCAASGSIYRINNMAWYRQAPGKGRELVSESTSGGNFIY
ADSVKGRFTISRDNSKNTLYLQMNSLRAEDTAVYYCAAFIIPSVGVFASRTYWGQGTLV
TVSS

The following table shows the amino acid sequences of CDR1, CDR2, and CDR3 of the respective clones.

| | | SEQ | | SEQ | | SEQ |
|---|---|---|---|---|---|---|
| | | ID | | ID | | ID |
| Clones | CDR1 | NO: | CDR2 | NO: | CDR3 | NO: |
| sdab-1 | IAVMA | 52 | AITFGGSTDYADAVKG | 53 | DVVTGLMRRDVY | 54 |
| sdab-2 | FMMG | 55 | TISSSGRTNYADSVKG | 56 | HYWRDY | 57 |
| sdab-3 | INNMA | 58 | EITSSDNTYYADSVKG | 59 | FIIPSVGVFSSRTY | 60 |
| sdab-4 | INTMG | 61 | AITTGGSTKYSDSVKG | 62 | DLKTNSNVFTWYEY | 63 |
| sdab-5 | FMGG | 64 | TISSSGRTNYADSVKG | 65 | HYWRDY | 66 |
| sdab-6 | IHNMG | 67 | STTNDGTTTYADSVKG | 68 | FIVTGFMASGDY | 69 |
| sdab-7 | TYAMS | 70 | STSSSGRTTSYSDSVKG | 71 | AGAYSGTDYYTLVRYYDY | 72 |
| sdab-8 | DYGMS | 73 | GISWRGGSTYYAESIKG | 74 | GSYYYNSRGYDY | 75 |
| sdab-9 | ISIMG | 76 | DISRGGKTNYADAVKA | 77 | QISNPFWGDY | 78 |
| sdab- | ITGMA | 79 | AIGSGSAGNTKYAESVKG | 80 | GLMTSQSYYSKPY | 81 |
| 10 | | | | | | |
| sdab- | IPLMA | 82 | GITTGGTTNYADSAKG | 83 | DVVTGLMRRNVY | 84 |
| 11 | | | | | | |
| sdab- | IDVMA | 85 | RIWADGSTTYADSMKG | 86 | VGTIADGAFRPYNN | 87 |
| 12 | | | | | | |
| sdab- | MASMA | 88 | LITFGGTTNYADSVKG | 89 | DVVTGLMRRDVY | 90 |
| 13 | | | | | | |
| sdab- | INNMA | 91 | EITSSGNTDYADSVKG | 92 | FIIPSVGVFTSRTY | 93 |
| 14 | | | | | | |
| sdab- | INSMS | 94 | TITVGGSTNYADSVKG | 95 | RIVSGFLRGPDDY | 96 |
| 15 | | | | | | |
| sdab- | ISGMG | 97 | AIGSGGDGDTKYAESVKG | 98 | GFMTSRNYYNGDY | 99 |
| 16 | | | | | | |
| sdab- | IGTMG | 100 | IATNGAIQNYAESVKG | 101 | DSQFGPWLRQYEY | 102 |
| 17 | | | | | | |
| sdab- | INAMG | 103 | DISRGGSTYYADSVKG | 104 | HDVPPFVPGGKSY | 105 |
| 18 | | | | | | |
| sdab- | ILAMG | 106 | TIHSRGNTNYADSVKG | 107 | HFWIDY | 108 |
| 19 | | | | | | |
| sdab- | IAVMG | 109 | TITRGGATHYADSTKG | 110 | RALIKVFHPPNDY | 111 |
| 20 | | | | | | |
| sdab- | LNDMG | 112 | SIFSSGTTNYADSAKG | 113 | DVVTGLLQRNVY | 114 |
| 21 | | | | | | |
| sdab- | IQTMG | 115 | DITTGGGSTYYVDSVKG | 116 | NVLEALFRPNLEV | 117 |
| 22 | | | | | | |
| sdab- | MVTMG | 118 | IITSGGSADYADSVKG | 119 | RVVADFQKVLDV | 120 |
| 23 | | | | | | |
| sdab- | LMTMG | 121 | TATPGGTTNYADSVKG | 122 | DVVYGLARQGNY | 123 |
| 24 | | | | | | |
| sdab- | INNMA | 124 | ESTSGGNFIYADSVKG | 125 | FIIPSVGVFASRTY | 126 |
| 25 | | | | | | |
| sdab- | INEMA | 127 | VAPNSVNPNYADSVKG | 128 | FIIPSVGVFTSRVY | 129 |
| 26 | | | | | | |
| sdab- | IVTMG | 130 | TTTRGGSTNYADSVKG | 131 | EIIPGFLQQLTLY | 132 |
| 27 | | | | | | |
| sdab- | DIRFMG | 133 | MVDTTGRINYADSVKG | 134 | VLWPRGAISAMTV | 135 |
| 28 | | | | | | |
| sdab- | INNMA | 136 | EITSSGNTLYADSVKG | 137 | FIIPSVGVFISRTF | 138 |
| 29 | | | | | | |
| sdab- | INNMA | 139 | EITSSGNTLYADSVKG | 140 | FIIPSVGVFTSRAY | 141 |
| 30 | | | | | | |
| sdab- | FVVMG | 142 | MLTSSGTTNYADSVKG | 143 | VIRSDSSWRADANEY | 144 |
| 31 | | | | | | |
| sdab- | STYMG | 145 | RIAGGGQINYADSVKG | 146 | HYWMDY | 147 |
| 32 | | | | | | |
| sdab- | INNMA | 148 | EITNSGNTLYTDSVKG | 149 | FIIPSVGVFTSRAY | 150 |
| 33 | | | | | | |
| sdab- | INTMG | 151 | IVGKYGETNYADSVKG | 152 | DVLAAGWWSGRVL | 153 |
| 34 | | | | | | |
| sdab- | INTMG | 154 | GITTGGTTRYADSVKG | 155 | DLKTTSNVFQWYEY | 156 |
| 35 | | | | | | |
| sdab- | NTYMG | 157 | RISGGGSVHYLDSVKG | 158 | HYWMDY | 159 |
| 36 | | | | | | |
| sdab | IVTMG | 160 | TTTRGGSTSYSDSVKG | 161 | DIIPGFLQQLTVH | 162 |
| 37 | | | | | | |
| sdab- | IPIMS | 163 | GISTGGSTNYADSVKG | 164 | LIKEEAWGTFGDY | 165 |
| 38 | | | | | | |
| sdab- | IVTMG | 166 | SITRGGSTNYADFVKG | 167 | DDVPPLTLTRVIY | 168 |
| 39 | | | | | | |
| sdab- | IYDMA | 169 | RIVWRSGGSTSYADSVKG | 170 | FEFPLQIGGQNVYY | 171 |
| 40 | | | | | | |
| sdab- | FVVMG | 172 | TLTSSGATTYADSVKG | 173 | VLRSGDNWMAAANEY | 174 |
| 41 | | | | | | |
| sdab- | TYDTN | 175 | FINYRGGGATYADSVKD | 176 | HERFPMGRPPYDY | 177 |
| 42 | | | | | | |
| sdab- | NYYMG | 178 | RINLSGSSIYYADSVKG | 179 | RDIVPGAQDY | 180 |
| 43 | | | | | | |
| sdab- | INTVA | 181 | EITSSGNRDYADSVKG | 182 | FIIPSVGLFTSRTY | 183 |
| 44 | | | | | | |
| sdab- | INYMA | 184 | RAWTTGPPTYAESVKG | 185 | NTLTSGY | 186 |
| 45 | | | | | | |
| sdab- | TDVMA | 187 | TLLGRSTTNYADSVKG | 188 | HRNLFNKIDNY | 189 |
| 46 | | | | | | |
| sdab- | IVTMG | 190 | TTTRGGTTNYSDSVKG | 191 | DIIPGFLQQLTVH | 192 |
| 47 | | | | | | |
| sdab- | VMVMG | 193 | TQTIGGTTNYADSVKG | 194 | DVVYGLARSGNY | 195 |
| 48 | | | | | | |
| sdab- | FVVMG | 196 | MLTSSGTTNYADSVKG | 197 | VLRSGSDWRAAANEY | 198 |
| 49 | | | | | | |
| sdab- | INAMA | 199 | EITSTGNTDYADSVKG | 200 | FIIPSVGMFTSRTY | 201 |
| 50 | | | | | | |

While the invention has been described above in connection with one or more embodiments, it should be understood that the invention is not limited to those embodiments, and the description is intended to cover all alternatives, modifications, and equivalents, as may be included within the spirit and scope of the appended claims.

All references cited herein are incorporated by reference in their entirety.

1. An isolated antibody, or an antigen-binding portion thereof, comprising:
a monomeric variable domain comprising a CDR1 region, a CDR2 region, and a CDR3 region comprising the amino acid sequences of:
(1) SEQ ID NOs: 199, 200, and 201, respectively;
(2) SEQ ID NOs: 55, 56, and 57, respectively;
(3) SEQ ID NOs: 58, 59, and 60, respectively;
(4) SEQ ID NOs: 61, 62, and 63, respectively;
(5) SEQ ID NOs: 64, 65, and 66, respectively;
(6) SEQ ID NOs: 76, 77, and 78, respectively;
(7) SEQ ID NOs: 82, 83, and 84, respectively;
(8) SEQ ID NOs: 85, 86, and 87, respectively;
(9) SEQ ID NOs: 88, 89, and 90, respectively;
(10) SEQ ID NOs: 91, 92, and 93, respectively;
(11) SEQ ID NOs: 94, 95, and 96, respectively;
(12) SEQ ID NOs: 103, 104, and 105, respectively;
(13) SEQ ID NOs: 106, 107, and 108, respectively;
(14) SEQ ID NOs: 112, 113, and 114, respectively;
(15) SEQ ID NOs: 115, 116, and 117, respectively;
(16) SEQ ID NOs: 121, 122, and 123, respectively;
(17) SEQ ID NOs: 124, 125, and 126, respectively;
(18) SEQ ID NOs: 127, 128, and 129, respectively;
(19) SEQ ID NOs: 133, 134, and 135, respectively;
(20) SEQ ID NOs: 136, 137, and 138, respectively;
(21) SEQ ID NOs: 139, 140, and 141, respectively;
(22) SEQ ID NOs: 142, 143, and 144, respectively;
(23) SEQ ID NOs: 145, 146, and 147, respectively;
(24) SEQ ID NOs: 148, 149, and 150, respectively;
(25) SEQ ID NOs: 151, 152, and 153, respectively;
(26) SEQ ID NOs: 154, 155, and 156, respectively;
(27) SEQ ID NOs: 157, 158, and 159, respectively;
(28) SEQ ID NOs: 160, 161, and 162, respectively;
(29) SEQ ID NOs: 163, 164, and 165, respectively;
(30) SEQ ID NOs: 169, 170, and 171, respectively;
(31) SEQ ID NOs: 172, 173, and 174, respectively;
(32) SEQ ID NOs: 175, 176, and 177, respectively;
(33) SEQ ID NOs: 178, 179, and 180, respectively;
(34) SEQ ID NOs: 181, 182, and 183, respectively;
(35) SEQ ID NOs: 184, 185, and 186, respectively;
(36) SEQ ID NOs: 190, 191, and 192, respectively;
(37) SEQ ID NOs: 193, 194, and 195, respectively;
(38) SEQ ID NOs: 196, 197, and 198, respectively;
(39) SEQ ID NOs: 52, 53, and 54, respectively; or
a variant thereof comprising up to about 3 amino acid substitutions in any one or more of the CDR1, CDR2, and CDR3.

2. The antibody or antigen-binding portion thereof, of claim 1, wherein the CDR1 region, the CDR2 region, and the CDR3 region comprising the amino acid sequences of:
SEQ ID NOs: 199, 200, and 201, respectively;
SEQ ID NOs: 76, 77, and 78, respectively;
SEQ ID NOs: 124, 125, and 126, respectively;
SEQ ID NOs: 136, 137, and 138, respectively;
SEQ ID NOs: 145, 146, and 147, respectively;
SEQ ID NOs: 175, 176, and 177, respectively;
SEQ ID NOs: 64, 65, and 66, respectively; or
a variant of any of the above comprising up to about 3 amino acid substitutions in any one or more of the CDR1, CDR2, and CDR3.

3. The antibody or antigen-binding portion thereof, of claim 1, wherein the CDR1 region, the CDR2 region, and the CDR3 region comprising the amino acid sequences of:
SEQ ID NOs: 199, 200, and 201, respectively;
SEQ ID NOs: 76, 77, and 78, respectively;
SEQ ID NOs: 124, 125, and 126, respectively;
SEQ ID NOs: 136, 137, and 138, respectively;
SEQ ID NOs: 145, 146, and 147, respectively;
SEQ ID NOs: 175, 176, and 177, respectively; or
SEQ ID NOs: 64, 65, and 66, respectively.

4. The antibody or an antigen-binding portion thereof, of claim 1, which specifically binds to Claudin 18.2, preferably human Claudin 18.2.

5. The antibody or antigen-binding portion thereof, of claim 1, which is a single domain antibody (sdAb) or a V_{H}H domain.

6. The antibody or antigen-binding portion thereof, of claim 1, which is camelid, chimeric, human or humanized.

7. The antibody or antigen-binding portion thereof, of claim 1, wherein the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs: 1, 2, 3, 4, 5, 9, 11, 12, 13, 14, 15, 18, 19, 21, 22, 24, 25, 26, 28, 29, 31, 32, 33, 34, 35, 36, 37, 38, 40, 41, 42, 43, 44, 45, 47, 48, 49, 50 and 202-213.

8. (canceled)

9. The antibody or antigen-binding portion thereof, of claim 1, wherein the monomeric variable domain comprises an amino acid sequence having at least 80%, 85%, 90%, 92%, 94%, 95%, 96%, 97%, 98%, or 99% identity to the amino acid sequence selected from the group consisting of SEQ ID NOs:5, 9, 25, 29, 32, 42, 50 and 202-213.

10. (canceled)

11. An antibody or antigen-binding portion thereof, comprising a monomeric variable domain comprising an amino acid sequence selected from the group consisting of SEQ ID NOs: 1, 2, 3, 4, 5, 9, 11, 12, 13, 14, 15, 18, 19, 21, 22, 24, 25, 26, 28, 29, 30, 31, 32, 33, 34, 35, 36, 37, 38, 40, 41, 42, 43, 44, 45, 47, 48, 49, 50 and 202-213.

12. The antibody or antigen-binding portion thereof, of claim 11, wherein the monomeric variable domain comprises the amino acid sequence selected from the group consisting of SEQ ID NOs: 5, 9, 25, 29, 32, 42, 50 and 202-213.

13. The antibody or antigen-binding portion thereof, of claim 1, further comprising a IgG Fc region fused with the monomeric variable domain.

14. The antibody or antigen-binding portion thereof, of claim 13, wherein the IgG Fc region is a human IgG1 Fc region comprising the amino acid sequence of SEQ ID NO: 51.

15. A bispecific molecule, an immunoconjugate, or a chimeric antigen receptor, comprising the antibody or antigen-binding portion thereof of claim 1.

16. A nucleic acid molecule encoding the antibody or antigen-binding portion thereof of claim 1.

17. An expression vector containing the nucleic acid molecule of claim 16.

18. A host cell containing the expression vector of claim 17.

19. A pharmaceutical composition comprising the antibody or antigen-binding portion thereof, of claim 1 and a pharmaceutically acceptable carrier.

20. The pharmaceutical composition of claim 19, further comprising a cytotoxic agent.

21. A method for treating a cancer disease in a subject, comprising administering to the subject a therapeutically effective amount of the pharmaceutical composition of claim 19.

22. The method of claim 21, wherein the cancer disease is selected from the group consisting of gastric cancer, pancreatic cancer, colon cancer, esophageal cancer, hepatic cancer, ovarian cancer, lung cancer and bladder cancer.


SEQUENCE LISTING (213 sequences)

SEQ ID NO: 1 - protein, 120 aa, Artificial Sequence
  sdab-1
QVKLEESGGGLAQAGGSLRLSCAASGRILNIAVMAWYRQTPGRQRELVAAITFGGSTDYADAVKGRFTISRDSAKNTVYLQMNDLKSEDTAVYYCNADVVTGLMRRDVYWGQGTQVTVSS

SEQ ID NO: 2 - protein, 111 aa, Artificial Sequence
  sdab-2
QVKLEESGGGLVQAGGSLRLSCAASVEIFMMGWYRQAPGKQRELVATISSSGRTNYADSVKGRFTISGDNSKNTVYLQMNSLKPEDTAVYYCYCHYWRDYWGRGTQVTVSS

SEQ ID NO: 3 - protein, 122 aa, Artificial Sequence
  sdab-3
QVKLEESGGGLVQAGGSLRLSCAASGSIYRINNMAWYRQAPGKQRELVAEITSSDNTYYADSVKGRFTISRDNAKNTVYLQMNSLKPEDTAVYSCNAFIIPSVGVFSSRTYWGRGTQVTVSS

SEQ ID NO: 4 - protein, 122 aa, Artificial Sequence
  sdab-4
QVQLVESGGGLVQPGGSLRLSCAASGSIFQINTMGWYRQAPGKQRELVAAITTGGSTKYSDSVKGRFTISRDNAKNTVYLQMNSLKPEDTAVYYCNADLKTNSNVFTWYEYWGQGTQVTVSS

SEQ ID NO: 5 - protein, 111 aa, Artificial Sequence
  sdab-5
DVQLVESGGGLVQAGGSLRLSCAASVGIFMGGWYRQAPGKQRELVATISSSGRTNYADSVKGRFTISGDNSMNTVYLQMNSLKPEDTAVYYCYAHYWRDYWGRGTQVTVSS

SEQ ID NO: 6 - protein, 120 aa, Artificial Sequence
  sdab-6
AVQLVDSGGGLVQAGGSLRLSCAPSGKIFSIHNMGWYRQAPGKQRELVASTTNDGTTTYADSVKGRFIISRDIAKNTVYLQMQSLKPEDTAVYYCNAFIVTGFMASGDYWGQGTQVTVSS

SEQ ID NO: 7 - protein, 127 aa, Artificial Sequence
  sdab-7
DVQLVESGGGLVQPGGSLRLSCAASGFTFSTYAMSWVRQAPGKGLEWVSSTSSSGRTTSYSDSVKGRFTISRDNAKSTLYLQMNSLKPEDTAVYYCAKAGAYSGTDYYTLVRYYDYWGQGTPVTVSS

SEQ ID NO: 8 - protein, 121 aa, Artificial Sequence
  sdab-8
DVQLVESGGGLVQPGGSLRLSCAASGFTFDDYGMSWVRQAPGKGLEWVSGISWRGGSTYYAESIKGRFTASRDNAKDTVYLQMNSLKPEDTAVYYCVRGSYYYNSRGYDYWGQGTPVTVSS

SEQ ID NO: 9 - protein, 118 aa, Artificial Sequence
  sdab-9
DVQLVESGGGLVQAGGSLTLSCAASRSDLFISIMGWYRQAPGKQRELVADISRGGKTNYADAVKARATISRDNAKNTMYLQMNNVKPEDTAVYYCYAQISNPFWGDYWGRGTQVTVSS

SEQ ID NO: 10 - protein, 123 aa, Artificial Sequence
  sdab-10
QVKLEESGGGFVQAGGSLRLSCSASGSIFGITGMAWYRQAPGKERELIAAIGSGSAGNTKYAESVKGRFTIFGDYAKKTLYLQMNSLKPEDTGVYYCNAGLMTSQSYYSKPYWGLGTQVTVSA

SEQ ID NO: 11 - protein, 120 aa, Artificial Sequence
  sdab-11
EVQLVESGGGLVQAGGSLRLSCVASGTILNIPLMAWFRQTPGKQRELVAGITTGGTTNYADSAKGRFTISRDNAKKTIYLQMDSLKPEDTAVYYCNADVVTGLMRRNVYWGQGTQVTVSS

SEQ ID NO: 12 - protein, 122 aa, Artificial Sequence
  sdab-12
QVKLEESGGGLVQAGGSLTLSCAASRSIFSIDVMAWYRQAPGKQRELVARIWADGSTTYADSMKGRFAISRDTAKNTVYLQMNSLKPEDTAVYYCNAVGTIADGAFRPYNNWGQGTQVTVSS

SEQ ID NO: 13 - protein, 120 aa, Artificial Sequence
  sdab-13
AVQLVESGGGLAQAGGSLRLSCAASGRILNMASMAWFRQTPGKQRESVALITFGGTTNYADSVKGRFTISRDNAKNTVYLQMNDLKSEDTAVYYCNADVVTGLMRRDVYWGQGTQVIVSS

SEQ ID NO: 14 - protein, 122 aa, Artificial Sequence
  sdab-14
QVKLEESGGGLVQAGGSLRLSCAASGSIYRINNMAWYRQAPGKQRELVAEITSSGNTDYADSVKGRFTISRDNAKNTVYLQMNSLKPEDTAVYSCNAFIIPSVGVFTSRTYWGRGTQVTVSS

SEQ ID NO: 15 - protein, 121 aa, Artificial Sequence
  sdab-15
QVKLEESGGGLVQAGGSLRLSCAASGSILNINSMSWYRQAPGKQRELVATITVGGSTNYADSVKGRFTISRYNANNMMYLQMNSLKPDDTGVYSCNARIVSGFLRGPDDYWGQGTQVTVSS

SEQ ID NO: 16 - protein, 123 aa, Artificial Sequence
  sdab-16
QVQLVESGGQSAQAGGSLRLSCTASGSIFGISGMGWYRQAPGKERELIAAIGSGGDGDTKYAESVKGRFTISGDYAKKTLYLQMNSLKPEDSGVYYCNAGFMTSRNYYNGDYWGQGTQVTVAA

SEQ ID NO: 17 - protein, 121 aa, Artificial Sequence
  sdab-17
EVQLVESGGGLVQAGGSLRLSCVWSGSFDDIGTMGWYRQAPGKQRELVAIATNGAIQNYAESVKGRFTVSRDNAKNTLYLQMNNLKPEDTAVYYCKMDSQFGPWLRQYEYWGQGTQVTVSP

SEQ ID NO: 18 - protein, 121 aa, Artificial Sequence
  sdab-18
AVQLVESGGGLVQAGGSLRLSCAASGSIFMINAMGWYRQAPGKQRELVADISRGGSTYYADSVKGRFTISRDNDKNTVSLQMNSLKPEDTAAYYCHAHDVPPFVPGGKSYWGQGTQVTVSS

SEQ ID NO: 19 - protein, 114 aa, Artificial Sequence
  sdab-19
QVKLEESGGGLVQAGGSLRLSCAASVSIDGILAMGWYRQAPGKQRELVATIHSRGNTNYADSVKGRFTVSRENNKNTMYLQMNSLKPEDTAVYYCYVHFWIDYWGQGTQVTVSS

SEQ ID NO: 20 - protein, 121 aa, Artificial Sequence
  sdab-20
QVKLEESGGGLVQAGGSLRLSCVASGSFLHIAVMGWYRQAPGKQRELVATITRGGATHYADSTKGRFSISSDNAKNAVALQMNSLQPDDTAVYFCNVRALIKVFHPPNDYWGQGTQVTVSS

SEQ ID NO: 21 - protein, 120 aa, Artificial Sequence
  sdab-21
QVQLVESGGGLVQAGGSLRLSCAASGRILNLNDMGWYRQTPGKQRELVASIFSSGTTNYADSAKGRFTISRDNAKKMIYLQMNSLKPEDTAVYYCNADVVTGLLQRNVYWGQGTQVTVSS

SEQ ID NO: 22 - protein, 122 aa, Artificial Sequence
  sdab-22
AVQLVESGGGLVQAGGSLRLSCVASGSWFRIQTMGWYRQTPGKQRELVADITTGGGSTYYVDSVKGRFTISRDNAKNTVYLQMINLKPEDTAAYYCQANVLEALFRPNLEVWGQGTLVTVSS

SEQ ID NO: 23 - protein, 120 aa, Artificial Sequence
  sdab-23
AVQLVDSGGGLVQAGGSLRLSCAASGIWLHMVTMGWYRQAPGKQRELVAIITSGGSADYADSVKGRFAISRDNTKNTVTLQMNSLKPDDTAVYFCNARVVADFQKVLDVWGQGTLVTVSS

SEQ ID NO: 24 - protein, 120 aa, Artificial Sequence
  sdab-24
QVKLEESGGGLVEAGGSLRLSCVASGSILNLMTMGWYRQAPGKQRELVATATPGGTTNYADSVKGRFTISRDNAKNTVYLQMNTLRPGDTAVYYCNADVVYGLARQGNYWGPGTQVTVSS

SEQ ID NO: 25 - protein, 122 aa, Artificial Sequence
  sdab-25
QVKLEESGGGLVQAGGSLRLSCVASGSIYRINNMAWYRQAPGKQRELVAESTSGGNFIYADSVKGRFAISRDNAKNTVYLQMNSLQPEDTAVYSCNAFIIPSVGVFASRTYWGRGTQVTVSS

SEQ ID NO: 26 - protein, 122 aa, Artificial Sequence
  sdab-26
DVQLVESGGGPVQAGGSLRLSCVASGSIYRINEMAWYRQAPGKQRELVAVAPNSVNPNYADSVKGRFTISRGNAKNTVYLQMNSLKPEDTAVYSCNAFIIPSVGVFTSRVYWGRGTQVTVSS

SEQ ID NO: 27 - protein, 121 aa, Artificial Sequence
  sdab-27
AVQLVDSGGGLVQAGGSLRLSCAVSGSFLNIVTMGWYRQAPGKQRELVATTTRGGSTNYADSVKGRFTIARDNAKNTVYLHMDALKPEDTAVYYCNAEIIPGFLQQLTLYWGQGTQVTVSA

SEQ ID NO: 28 - protein, 126 aa, Artificial Sequence
  sdab-28
QVKLEESGGGLVQAGGSLRLSCAASGSSNADIRFMGWYRQPAGNQRNNQREPVAMVDTTGRINYADSVKGRFTISRDNAKNTVSLQMNSLKPEDTALYYCTAVLWPRGAISAMTVWGQGTQVTVSS

SEQ ID NO: 29 - protein, 122 aa, Artificial Sequence
  sdab-29
QVKLEESGGGLVQAGGSLRLSCAASGSIYRINNMAWYRQIPGKQRELVAEITSSGNTLYADSVKGRFTISMDNAKNTAYLQMNSLKPEDTAVYSCNAFIIPSVGVFISRTFWGRGTQVTVSS

SEQ ID NO: 30 - protein, 122 aa, Artificial Sequence
  sdab-30
QVQLVESGGGLVQAGGSLRLSCAASGSIYRINNMAWYRQAPGKQRELVAEITSSGNTLYADSVKGRFTISSDGARNTVYLQMNSLKPEDTAVYSCNAFIIPSVGVFTSRAYWGRGTQVTVSS

SEQ ID NO: 31 - protein, 123 aa, Artificial Sequence
  sdab-31
AVQLVESGGDLVQAGGSLRLSCAASGSIFGFVVMGWYRQAPGNQRELVAMLTSSGTTNYADSVKGRFTLFRENNKNTVHLQMNSLKPEDTGVYYCRAVIRSDSSWRADANEYWGQGTHVTVSS

SEQ ID NO: 32 - protein, 114 aa, Artificial Sequence
  sdab-32
QVKLEESGGGLVQAGGSLRLSCAIPGSIVGSTYMGWFRQAPGKQRELVARIAGGGQINYADSVKGRFTISRDNAKNSLYLQMNSLTPEDTAVYYCYVHYWMDYWGQGTQVTVSS

SEQ ID NO: 33 - protein, 122 aa, Artificial Sequence
  sdab-33
QVKLEESGGGLVQAGGSLRLSCAASGSIYRINNMAWYRQAPGKQRELVAEITNSGNTLYTDSVKGRFTISMDGAKNAVYLQMNSLKPEDTAVYSCNAFIIPSVGVFTSRAYWGRGTQVTVSS

SEQ ID NO: 34 - protein, 121 aa, Artificial Sequence
  sdab-34
QVKLEESGGGLVQTGGSLSLSCVASGSGISINTMGWYRQGPGKQRELVAIVGKYGETNYADSVKGRFTISRDDAKKTVYLRMNSLKPEDTAVYYCNADVLAAGWWSGRVLWGQGTQVTVSS

SEQ ID NO: 35 - protein, 122 aa, Artificial Sequence
  sdab-35
AVQLVESGGGLVQAGGSLRLSCVASGSIFQINTMGWYRQAPGKQREWVTGITTGGTTRYADSVKGRFTISRDNAKNTVYLQMNSLKPEDTAVYYCNSDLKTTSNVFQWYEYWGQGTQVTVSA

SEQ ID NO: 36 - protein, 114 aa, Artificial Sequence
  sdab-36
QVKLEESGGGLVQAGGSLRLSCAISGSIFSNTYMGWYRQAPGRQRELVARISGGGSVHYLDSVKGRFTISRDNAKNTLNLQMNSLTPEDTAIYYCYAHYWMDYWGQGTQVTVSS

SEQ ID NO: 37 - protein, 121 aa, Artificial Sequence
  sdab-37
QVKLEESGGGLVQAGGSLRLSCAVSGNFLNIVTMGWYRQAPGKQRELVATTTRGGSTSYSDSVKGRFTISRDNAKSTVYLQMNSLKPEDTAVYYCNADIIPGFLQQLTVHWGQGTRVTVSS

SEQ ID NO: 38 - protein, 121 aa, Artificial Sequence
  sdab-38
EVQLVESGGGLVQAGGSLRLSCVASGSIFHIPIMSWYRRAPGKQRELVAGISTGGSTNYADSVKGRFTISRDNAKNTVYLQMNSLKPDDTAVYYCNSLIKEEAWGTFGDYWGQGTQVTVSS

SEQ ID NO: 39 - protein, 121 aa, Artificial Sequence
  sdab-39
QVKLEESGGGLAEAGGSLRLSCVASGSIFMIVTMGWYRQAPGKQRELVASITRGGSTNYADFVKGRFTISRDNAKNTVYLQMNSLKPEDTAVYFCNADDVPPLTLTRVIYWGQGTQVTVSS

SEQ ID NO: 40 - protein, 124 aa, Artificial Sequence
  sdab-40
QVQLVESGGGLVQAGGSLRVSCAASGRTSSIYDMAWYRQAPGNEREFVARIVWRSGGSTSYADSVKGRFTISRNTAKNTVYLQMNSLKPEDTAMYYCRAFEFPLQIGGQNVYYWGQGTQVTVSS

SEQ ID NO: 41 - protein, 123 aa, Artificial Sequence
  sdab-41
QVQLVESGGDLVQAGGSLRLSCAASGSPFGFVVMGWYRQAPGKQRDMVATLTSSGATTYADSVKGRFTLSRENAKNTIHLQMNSLKPEDTAVYYCRAVLRSGDNWMAAANEYWGQGTHVTVSS

SEQ ID NO: 42 - protein, 122 aa, Artificial Sequence
  sdab-42
EVQLVESGGGLVQAGGSLRLACAASGRAIGTYDTNWYRQAPGQQREFVAFINYRGGGATYADSVKDRFTISRDNAANTVYLQMNSLKPEDTAIYLCNAHERFPMGRPPYDYWGQGTQVTVTS

SEQ ID NO: 43 - protein, 119 aa, Artificial Sequence
  sdab-43
EVQLVESGGGLVQAGGSLRLSCEASGPARNNYYMGWFRQAPGKERELVARINLSGSSIYYADSVKGRFTISRDNAKNTMSLQMNSLKPGDAARYYCHARDIVPGAQDYWGQGTQVTVSA

SEQ ID NO: 44 - protein, 122 aa, Artificial Sequence
  sdab-44
EVQLVESGGGLVQAGGSLRLSCAASGSIYRINTVAWYRQAPGKQRELVAEITSSGNRDYADSVKGRFTISRDNAKNTVYLQMNSLKPEDTAVYSCNAFIIPSVGLFTSRTYWGRGTQVTVSS

SEQ ID NO: 45 - protein, 115 aa, Artificial Sequence
  sdab-45
QVKLEESGGGLVQAGGSLRLSCAASGSISGINYMAWYRQAPGKQRDLVARAWTTGPPTYAESVKGRFTISRDNAKNTVYLQMNNLKPEDTGVYYCTANTLTSGYWGQGIQVTVSS

SEQ ID NO: 46 - protein, 119 aa, Artificial Sequence
  sdab-46
AVQLVDSGGGLVQAGGSLRLSCAASPTIFVTDVMAWYRQAPGNQRELVATLLGRSTTNYADSVKGRFTISRDNAKNTVYLQMNTLKPEDTAVYYCNLHRNLFNKIDNYWGQGTQVTVSS

SEQ ID NO: 47 - protein, 121 aa, Artificial Sequence
  sdab-47
QVKLEESGGGLVQAGGSLRLSCAVSGNFLNIVTMGWYRQAPGKQRELVATTTRGGTTNYSDSVKGRFTISRDNAKNTVYLQMNSLKPEDTAVYYCNADIIPGFLQQLTVHWGQGAQVTVSS

SEQ ID NO: 48 - protein, 120 aa, Artificial Sequence
  sdab-48
QVKLEESGGGLVQAGGSLGLSCAASGTILHVMVMGWYRQAPGKQRELVATQTIGGTTNYADSVKGRFTISRDNAKNTVYLQMNTLTPGDTAVYYCNADVVYGLARSGNYWGPGTQVTVSS

SEQ ID NO: 49 - protein, 123 aa, Artificial Sequence
  sdab-49
QVQLVESGGDLVQAGGSLRLSCAASGSPFGFVVMGWHRQAPGKQRELVAMLTSSGTTNYADSVKGRFTLSRENTKNTVHLQMNKLKPEDTAVYYCRAVLRSGSDWRAAANEYWGQGTHVTVSS

SEQ ID NO: 50 - protein, 122 aa, Artificial Sequence
  sdab-50
QVKLEESGGGLVQAGGSLRLSCAASGSIYRINAMAWYRQAPGKQREYVAEITSTGNTDYADSVKGRFTISRDNAENTVYLQMNSLKPEDTAVYSCNAFIIPSVGMFTSRTYWGRGTQVTVSS

SEQ ID NO: 51 - protein, 232 aa, Artificial Sequence
  Human IgG1 Fc region
EPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 5 aa, Artificial Sequence
  sdab-1 CDR1
IAVMA

SEQ ID NO: 53 - protein, 16 aa, Artificial Sequence
  sdab-1 CDR2
AITFGGSTDYADAVKG

SEQ ID NO: 54 - protein, 12 aa, Artificial Sequence
  sdab-1 CDR3
DVVTGLMRRDVY

SEQ ID NO: 55 - protein, 4 aa, Artificial Sequence
  sdab-2 CDR1
FMMG

SEQ ID NO: 56 - protein, 16 aa, Artificial Sequence
  sdab-2 CDR2
TISSSGRTNYADSVKG

SEQ ID NO: 57 - protein, 6 aa, Artificial Sequence
  sdab-2 CDR3
HYWRDY

SEQ ID NO: 58 - protein, 5 aa, Artificial Sequence
  sdab-3 CDR1
INNMA

SEQ ID NO: 59 - protein, 16 aa, Artificial Sequence
  sdab-3 CDR2
EITSSDNTYYADSVKG

SEQ ID NO: 60 - protein, 14 aa, Artificial Sequence
  sdab-3 CDR3
FIIPSVGVFSSRTY

SEQ ID NO: 61 - protein, 5 aa, Artificial Sequence
  sdab-4 CDR1
INTMG

SEQ ID NO: 62 - protein, 16 aa, Artificial Sequence
  sdab-4 CDR2
AITTGGSTKYSDSVKG

SEQ ID NO: 63 - protein, 14 aa, Artificial Sequence
  sdab-4 CDR3
DLKTNSNVFTWYEY

SEQ ID NO: 64 - protein, 4 aa, Artificial Sequence
  sdab-5 CDR1
FMGG

SEQ ID NO: 65 - protein, 16 aa, Artificial Sequence
  FMGG
TISSSGRTNYADSVKG

SEQ ID NO: 66 - protein, 6 aa, Artificial Sequence
  sdab-5 CDR3
HYWRDY

SEQ ID NO: 67 - protein, 5 aa, Artificial Sequence
  sdab-6 CDR1
IHNMG

SEQ ID NO: 68 - protein, 16 aa, Artificial Sequence
  sdab-6 CDR2
STTNDGTTTYADSVKG

SEQ ID NO: 69 - protein, 12 aa, Artificial Sequence
  sdab-6 CDR3
FIVTGFMASGDY

SEQ ID NO: 70 - protein, 5 aa, Artificial Sequence
  sdab-7 CDR1
TYAMS

SEQ ID NO: 71 - protein, 17 aa, Artificial Sequence
  sdab-7 CDR2
STSSSGRTTSYSDSVKG

SEQ ID NO: 72 - protein, 18 aa, Artificial Sequence
  sdab-7 CDR3
AGAYSGTDYYTLVRYYDY

SEQ ID NO: 73 - protein, 5 aa, Artificial Sequence
  sdab-8 CDR1
DYGMS

SEQ ID NO: 74 - protein, 17 aa, Artificial Sequence
  sdab-8 CDR2
GISWRGGSTYYAESIKG

SEQ ID NO: 75 - protein, 12 aa, Artificial Sequence
  sdab-8 CDR3
GSYYYNSRGYDY

SEQ ID NO: 76 - protein, 5 aa, Artificial Sequence
  sdab-9 CDR1
ISIMG

SEQ ID NO: 77 - protein, 16 aa, Artificial Sequence
  sdab-9 CDR2
DISRGGKTNYADAVKA

SEQ ID NO: 78 - protein, 10 aa, Artificial Sequence
  sdab-9 CDR3
QISNPFWGDY

SEQ ID NO: 79 - protein, 5 aa, Artificial Sequence
  sdab-10 CDR1
ITGMA

SEQ ID NO: 80 - protein, 18 aa, Artificial Sequence
  sdab-10 CDR2
AIGSGSAGNTKYAESVKG

SEQ ID NO: 81 - protein, 13 aa, Artificial Sequence
  sdab-10 CDR3
GLMTSQSYYSKPY

SEQ ID NO: 82 - protein, 5 aa, Artificial Sequence
  sdab-11 CDR1
IPLMA

SEQ ID NO: 83 - protein, 16 aa, Artificial Sequence
  sdab-11 CDR2
GITTGGTTNYADSAKG

SEQ ID NO: 84 - protein, 12 aa, Artificial Sequence
  sdab-11 CDR3
DVVTGLMRRNVY

SEQ ID NO: 85 - protein, 5 aa, Artificial Sequence
  sdab-12 CDR1
IDVMA

SEQ ID NO: 86 - protein, 16 aa, Artificial Sequence
  sdab-12 CDR2
RIWADGSTTYADSMKG

SEQ ID NO: 87 - protein, 14 aa, Artificial Sequence
  sdab-12 CDR3
VGTIADGAFRPYNN

SEQ ID NO: 88 - protein, 5 aa, Artificial Sequence
  sdab-13 CDR1
MASMA

SEQ ID NO: 89 - protein, 16 aa, Artificial Sequence
  sdab-13 CDR2
LITFGGTTNYADSVKG

SEQ ID NO: 90 - protein, 12 aa, Artificial Sequence
  sdab-13 CDR3
DVVTGLMRRDVY

SEQ ID NO: 91 - protein, 5 aa, Artificial Sequence
  sdab-14 CDR1
INNMA

SEQ ID NO: 92 - protein, 16 aa, Artificial Sequence
  sdab-14 CDR2
EITSSGNTDYADSVKG

SEQ ID NO: 93 - protein, 14 aa, Artificial Sequence
  sdab-14 CDR3
FIIPSVGVFTSRTY

SEQ ID NO: 94 - protein, 5 aa, Artificial Sequence
  sdab-15 CDR1
INSMS

SEQ ID NO: 95 - protein, 16 aa, Artificial Sequence
  sdab-15 CDR2
TITVGGSTNYADSVKG

SEQ ID NO: 96 - protein, 13 aa, Artificial Sequence
  sdab-15 CDR3
RIVSGFLRGPDDY

SEQ ID NO: 97 - protein, 5 aa, Artificial Sequence
  sdab-16 CDR1
ISGMG

SEQ ID NO: 98 - protein, 18 aa, Artificial Sequence
  sdab-16 CDR2
AIGSGGDGDTKYAESVKG

SEQ ID NO: 99 - protein, 13 aa, Artificial Sequence
  sdab-16 CDR3
GFMTSRNYYNGDY

SEQ ID NO: 100 - protein, 5 aa, Artificial Sequence
  sdab-17 CDR1
IGTMG

SEQ ID NO: 101 - protein, 16 aa, Artificial Sequence
  sdab-17 CDR2
IATNGAIQNYAESVKG

SEQ ID NO: 102 - protein, 13 aa, Artificial Sequence
  sdab-17 CDR3
DSQFGPWLRQYEY

SEQ ID NO: 103 - protein, 5 aa, Artificial Sequence
  sdab-18 CDR1
INAMG

SEQ ID NO: 104 - protein, 16 aa, Artificial Sequence
  sdab-18 CDR2
DISRGGSTYYADSVKG

SEQ ID NO: 105 - protein, 13 aa, Artificial Sequence
  sdab-18 CDR3
HDVPPFVPGGKSY

SEQ ID NO: 106 - protein, 5 aa, Artificial Sequence
  sdab-19 CDR1
ILAMG

SEQ ID NO: 107 - protein, 16 aa, Artificial Sequence
  sdab-19 CDR2
TIHSRGNTNYADSVKG

SEQ ID NO: 108 - protein, 6 aa, Artificial Sequence
  sdab-19 CDR3
HFWIDY

SEQ ID NO: 109 - protein, 5 aa, Artificial Sequence
  sdab-20 CDR1
IAVMG

SEQ ID NO: 110 - protein, 16 aa, Artificial Sequence
  sdab-20 CDR2
TITRGGATHYADSTKG

SEQ ID NO: 111 - protein, 13 aa, Artificial Sequence
  sdab-20 CDR3
RALIKVFHPPNDY

SEQ ID NO: 112 - protein, 5 aa, Artificial Sequence
  sdab-21 CDR1
LNDMG

SEQ ID NO: 113 - protein, 16 aa, Artificial Sequence
  sdab-21 CDR2
SIFSSGTTNYADSAKG

SEQ ID NO: 114 - protein, 12 aa, Artificial Sequence
  sdab-21 CDR3
DVVTGLLQRNVY

SEQ ID NO: 115 - protein, 5 aa, Artificial Sequence
  sdab-22 CDR1
IQTMG

SEQ ID NO: 116 - protein, 17 aa, Artificial Sequence
  sdab-22 CDR2
DITTGGGSTYYVDSVKG

SEQ ID NO: 117 - protein, 13 aa, Artificial Sequence
  sdab-22 CDR3
NVLEALFRPNLEV

SEQ ID NO: 118 - protein, 5 aa, Artificial Sequence
  sdab-23 CDR1
MVTMG

SEQ ID NO: 119 - protein, 16 aa, Artificial Sequence
  sdab-23 CDR2
IITSGGSADYADSVKG

SEQ ID NO: 120 - protein, 12 aa, Artificial Sequence
  sdab-23 CDR3
RVVADFQKVLDV

SEQ ID NO: 121 - protein, 5 aa, Artificial Sequence
  sdab-24 CDR1
LMTMG

SEQ ID NO: 122 - protein, 16 aa, Artificial Sequence
  sdab-24 CDR2
TATPGGTTNYADSVKG

SEQ ID NO: 123 - protein, 12 aa, Artificial Sequence
  sdab-24 CDR3
DVVYGLARQGNY

SEQ ID NO: 124 - protein, 5 aa, Artificial Sequence
  sdab-25 CDR1
INNMA

SEQ ID NO: 125 - protein, 16 aa, Artificial Sequence
  sdab-25 CDR2
ESTSGGNFIYADSVKG

SEQ ID NO: 126 - protein, 14 aa, Artificial Sequence
  sdab-25 CDR3
FIIPSVGVFASRTY

SEQ ID NO: 127 - protein, 5 aa, Artificial Sequence
  sdab-26 CDR1
INEMA

SEQ ID NO: 128 - protein, 16 aa, Artificial Sequence
  sdab-26 CDR2
VAPNSVNPNYADSVKG

SEQ ID NO: 129 - protein, 14 aa, Artificial Sequence
  sdab-26 CDR3
FIIPSVGVFTSRVY

SEQ ID NO: 130 - protein, 5 aa, Artificial Sequence
  sdab-27 CDR1
IVTMG

SEQ ID NO: 131 - protein, 16 aa, Artificial Sequence
  sdab-27 CDR2
TTTRGGSTNYADSVKG

SEQ ID NO: 132 - protein, 13 aa, Artificial Sequence
  sdab-27 CDR3
EIIPGFLQQLTLY

SEQ ID NO: 133 - protein, 6 aa, Artificial Sequence
  sdab-28 CDR1
DIRFMG

SEQ ID NO: 134 - protein, 16 aa, Artificial Sequence
  sdab-28 CDR2
MVDTTGRINYADSVKG

SEQ ID NO: 135 - protein, 13 aa, Artificial Sequence
  sdab-28 CDR3
VLWPRGAISAMTV

SEQ ID NO: 136 - protein, 5 aa, Artificial Sequence
  sdab-29 CDR1
INNMA

SEQ ID NO: 137 - protein, 16 aa, Artificial Sequence
  sdab-29 CDR2
EITSSGNTLYADSVKG

SEQ ID NO: 138 - protein, 14 aa, Artificial Sequence
  sdab-29 CDR3
FIIPSVGVFISRTF

SEQ ID NO: 139 - protein, 5 aa, Artificial Sequence
  sdab-30 CDR1
INNMA

SEQ ID NO: 140 - protein, 16 aa, Artificial Sequence
  sdab-30 CDR2
EITSSGNTLYADSVKG

SEQ ID NO: 141 - protein, 14 aa, Artificial Sequence
  sdab-30 CDR3
FIIPSVGVFTSRAY

SEQ ID NO: 142 - protein, 5 aa, Artificial Sequence
  sdab-31 CDR1
FVVMG

SEQ ID NO: 143 - protein, 16 aa, Artificial Sequence
  sdab-31 CDR2
MLTSSGTTNYADSVKG

SEQ ID NO: 144 - protein, 15 aa, Artificial Sequence
  sdab-31 CDR3
VIRSDSSWRADANEY

SEQ ID NO: 145 - protein, 5 aa, Artificial Sequence
  sdab-32 CDR1
STYMG

SEQ ID NO: 146 - protein, 16 aa, Artificial Sequence
  sdab-32 CDR2
RIAGGGQINYADSVKG

SEQ ID NO: 147 - protein, 6 aa, Artificial Sequence
  sdab-32 CDR3
HYWMDY

SEQ ID NO: 148 - protein, 5 aa, Artificial Sequence
  sdab-33 CDR1
INNMA

SEQ ID NO: 149 - protein, 16 aa, Artificial Sequence
  sdab-33 CDR2
EITNSGNTLYTDSVKG

SEQ ID NO: 150 - protein, 14 aa, Artificial Sequence
  sdab-33 CDR3
FIIPSVGVFTSRAY

SEQ ID NO: 151 - protein, 5 aa, Artificial Sequence
  sdab-34 CDR1
INTMG

SEQ ID NO: 152 - protein, 16 aa, Artificial Sequence
  sdab-34 CDR2
IVGKYGETNYADSVKG

SEQ ID NO: 153 - protein, 13 aa, Artificial Sequence
  sdab-34 CDR3
DVLAAGWWSGRVL

SEQ ID NO: 154 - protein, 5 aa, Artificial Sequence
  sdab-35 CDR1
INTMG

SEQ ID NO: 155 - protein, 16 aa, Artificial Sequence
  sdab-35 CDR2
GITTGGTTRYADSVKG

SEQ ID NO: 156 - protein, 14 aa, Artificial Sequence
  sdab-35 CDR3
DLKTTSNVFQWYEY

SEQ ID NO: 157 - protein, 5 aa, Artificial Sequence
  sdab-36 CDR1
NTYMG

SEQ ID NO: 158 - protein, 16 aa, Artificial Sequence
  sdab-36 CDR2
RISGGGSVHYLDSVKG

SEQ ID NO: 159 - protein, 6 aa, Artificial Sequence
  sdab-36 CDR3
HYWMDY

SEQ ID NO: 160 - protein, 5 aa, Artificial Sequence
  sdab-37 CDR1
IVTMG

SEQ ID NO: 161 - protein, 16 aa, Artificial Sequence
  sdab-37 CDR2
TTTRGGSTSYSDSVKG

SEQ ID NO: 162 - protein, 13 aa, Artificial Sequence
  sdab-37 CDR3
DIIPGFLQQLTVH

SEQ ID NO: 163 - protein, 5 aa, Artificial Sequence
  sdab-38 CDR1
IPIMS

SEQ ID NO: 164 - protein, 16 aa, Artificial Sequence
  sdab-38 CDR2
GISTGGSTNYADSVKG

SEQ ID NO: 165 - protein, 13 aa, Artificial Sequence
  sdab-38 CDR3
LIKEEAWGTFGDY

SEQ ID NO: 166 - protein, 5 aa, Artificial Sequence
  sdab-39 CDR1
IVTMG

SEQ ID NO: 167 - protein, 16 aa, Artificial Sequence
  sdab-39 CDR2
SITRGGSTNYADFVKG

SEQ ID NO: 168 - protein, 13 aa, Artificial Sequence
  sdab-39 CDR3
DDVPPLTLTRVIY

SEQ ID NO: 169 - protein, 5 aa, Artificial Sequence
  sdab-40 CDR1
IYDMA

SEQ ID NO: 170 - protein, 18 aa, Artificial Sequence
  sdab-40 CDR2
RIVWRSGGSTSYADSVKG

SEQ ID NO: 171 - protein, 14 aa, Artificial Sequence
  sdab-40 CDR3
FEFPLQIGGQNVYY

SEQ ID NO: 172 - protein, 5 aa, Artificial Sequence
  sdab-41 CDR1
FVVMG

SEQ ID NO: 173 - protein, 16 aa, Artificial Sequence
  sdab-41 CDR2
TLTSSGATTYADSVKG

SEQ ID NO: 174 - protein, 15 aa, Artificial Sequence
  sdab-41 CDR3
VLRSGDNWMAAANEY

SEQ ID NO: 175 - protein, 5 aa, Artificial Sequence
  sdab-42 CDR1
TYDTN

SEQ ID NO: 176 - protein, 17 aa, Artificial Sequence
  sdab-42 CDR2
FINYRGGGATYADSVKD

SEQ ID NO: 177 - protein, 13 aa, Artificial Sequence
  sdab-42 CDR3
HERFPMGRPPYDY

SEQ ID NO: 178 - protein, 5 aa, Artificial Sequence
  sdab-43 CDR1
NYYMG

SEQ ID NO: 179 - protein, 17 aa, Artificial Sequence
  sdab-43 CDR2
RINLSGSSIYYADSVKG

SEQ ID NO: 180 - protein, 10 aa, Artificial Sequence
  sdab-43 CDR3
RDIVPGAQDY

SEQ ID NO: 181 - protein, 5 aa, Artificial Sequence
  sdab-44 CDR1
INTVA

SEQ ID NO: 182 - protein, 16 aa, Artificial Sequence
  sdab-44 CDR2
EITSSGNRDYADSVKG

SEQ ID NO: 183 - protein, 14 aa, Artificial Sequence
  sdab-44 CDR3
FIIPSVGLFTSRTY

SEQ ID NO: 184 - protein, 5 aa, Artificial Sequence
  sdab-45 CDR1
INYMA

SEQ ID NO: 185 - protein, 16 aa, Artificial Sequence
  sdab-45 CDR2
RAWTTGPPTYAESVKG

SEQ ID NO: 186 - protein, 7 aa, Artificial Sequence
  sdab-45 CDR3
NTLTSGY

SEQ ID NO: 187 - protein, 5 aa, Artificial Sequence
  sdab-46 CDR1
TDVMA

SEQ ID NO: 188 - protein, 16 aa, Artificial Sequence
  sdab-46 CDR2
TLLGRSTTNYADSVKG

SEQ ID NO: 189 - protein, 11 aa, Artificial Sequence
  sdab-46 CDR3
HRNLFNKIDNY

SEQ ID NO: 190 - protein, 5 aa, Artificial Sequence
  sdab-47 CDR1
IVTMG

SEQ ID NO: 191 - protein, 16 aa, Artificial Sequence
  sdab-47 CDR2
TTTRGGTTNYSDSVKG

SEQ ID NO: 192 - protein, 13 aa, Artificial Sequence
  sdab-47 CDR3
DIIPGFLQQLTVH

SEQ ID NO: 193 - protein, 5 aa, Artificial Sequence
  sdab-48 CDR1
VMVMG

SEQ ID NO: 194 - protein, 16 aa, Artificial Sequence
  sdab-48 CDR2
TQTIGGTTNYADSVKG

SEQ ID NO: 195 - protein, 12 aa, Artificial Sequence
  sdab-48 CDR3
DVVYGLARSGNY

SEQ ID NO: 196 - protein, 5 aa, Artificial Sequence
  sdab-49 CDR1
FVVMG

SEQ ID NO: 197 - protein, 16 aa, Artificial Sequence
  sdab-49 CDR2
MLTSSGTTNYADSVKG

SEQ ID NO: 198 - protein, 15 aa, Artificial Sequence
  sdab-49 CDR3
VLRSGSDWRAAANEY

SEQ ID NO: 199 - protein, 5 aa, Artificial Sequence
  sdab-50 CDR1
INAMA

SEQ ID NO: 200 - protein, 16 aa, Artificial Sequence
  sdab-50 CDR2
EITSTGNTDYADSVKG

SEQ ID NO: 201 - protein, 14 aa, Artificial Sequence
  sdab-50 CDR3
FIIPSVGMFTSRTY

SEQ ID NO: 202 - protein, 118 aa, Artificial Sequence
  CLDN18.2-vhh09-1.3
EVQLVESGGGLVQPGGSLRLSCAASRSDLFISIMGWYRQAPGKGREWVSDISRGGKTNYADAVKARFTISRDNSKNTLYLQMNSLRAEDTAVYYCAAQISNPFWGDYWGQGTLVTVSS

SEQ ID NO: 203 - protein, 122 aa, Artificial Sequence
  CLDN18.2-vhh42-1.1
QVQLVESGGGVVQPGGSLRLSCAASGRAIGTYDTNWYRQAPGKGREFVAFINYRGGGATYADSVKDRFTISRDNSKNTLYLQMNSLRAEDTAVYYCAAHERFPMGRPPYDYWGQGTLVTVSS

SEQ ID NO: 204 - protein, 122 aa, Artificial Sequence
  CLDN18.2-vhh42-2.2
QVQLVESGGGVVQPGGSLRLSCAASGFTFSTYDTNWYRQAPGKGREFVAFINYRGGGATYADSVKDRFTISRDNSKNTLYLQMNSLRAEDTAVYYCAAHERFPMGRPPYDYWGQGTLVTVSS

SEQ ID NO: 205 - protein, 122 aa, Artificial Sequence
  CLDN18.2-vhh50-1.1  & CLDN18.2-vhh50-2.1
EVQLVESGGGLVQPGGSLRLSCAASGSIYRINAMAWYRQAPGKGREYVSEITSTGNTDYADSVKGRFTISRDNSKNTLYLQMNSLRAEDTAVYYCAAFIIPSVGMFTSRTYWGQGTLVTVSS

SEQ ID NO: 206 - protein, 122 aa, Artificial Sequence
  CLDN18.2-vhh50-2.3
EVQLVESGGGLVQPGGSLRLSCAASGSIYRINAMAWYRQAPGKGREWVSEITSTGNTDYADSVKGRFTISRDNSKNTLYLQMNSLRAEDTAVYYCAAFIIPSVGMFTSRTYWGQGTLVTVSS

SEQ ID NO: 207 - protein, 122 aa, Artificial Sequence
  CLDN18.2-vhh29-1.1
EVQLVESGGGLIQPGGSLRLSCAASGSIYRINNMAWYRQAPGKGRELVSEITSSGNTLYADSVKGRFTISRDNSKNTLYLQMNSLRAEDTAVYYCAAFIIPSVGVFISRTFWGQGTTVTVSS

SEQ ID NO: 208 - protein, 122 aa, Artificial Sequence
  CLDN18.2-vhh29-2.1
QVQLVESGGGLVKPGGSLRLSCAASGSIYRINNMAWYRQAPGKGRELVSEITSSGNTLYADSVKGRFTISRDNAKNSLYLQMNSLRAEDTAVYYCAAFIIPSVGVFISRTFWGQGTLVTVSS

SEQ ID NO: 209 - protein, 122 aa, Artificial Sequence
  CLDN18.2-vhh29-2.3
QVQLVESGGGLVKPGGSLRLSCAASGSIYRINNMAWYRQAPGKGREWVSEITSSGNTLYADSVKGRFTISRDNAKNSLYLQMNSLRAEDTAVYYCAAFIIPSVGVFISRTFWGQGTLVTVSS

SEQ ID NO: 210 - protein, 122 aa, Artificial Sequence
  CLDN18.2-vhh29-3.1
EVQLVESGGGLVQPGGSLRLSCAASGSIYRINNMAWYRQAPGKGRELVSEITSSGNTLYADSVKGRFTISRDNSKNTLYLQMNSLRAEDTAVYYCAAFIIPSVGVFISRTFWGQGTLVTVSS

SEQ ID NO: 211 - protein, 114 aa, Artificial Sequence
  CLDN18.2-vhh32-1.2
EVQLVESGGGLVQPGGSLRLSCAASGFTVSSTYMGWFRQAPGKGRELVSRIAGGGQINYADSVKGRFTISRDNSKNTLYLQMNSLRAEDTAVYYCAAHYWMDYWGQGTLVTVSS

SEQ ID NO: 212 - protein, 122 aa, Artificial Sequence
  CLDN18.2-vhh25-1.1
EVQLVESGGGLVQPGGSLRLSCAASGSIYRINNMAWYRQAPGKGRELVSESTSGGNFIYADSVKGRFTISRDNSKNTLYLQMNSLRAEDTAVYYCAAFIIPSVGVFASRTYWGQGTLVTVSS

SEQ ID NO: 213 - protein, 122 aa, Artificial Sequence; identical to SEQ ID NO: 212
  CLDN18.2-vhh25-2.1